(19) United States
(12) Patent Application Publication
(10) Pub. No.: US 2012/0089117 A1
(43) Pub. Date: Apr. 12, 2012

(54) APPARATUS THAT INCLUDES NANO-SIZED PROJECTIONS AND A METHOD FOR MANUFACTURE THEREOF

(75) Inventors: Hans Junginger, Singapore (SG); Giorgia Pastorin, Singapore (SG); Minrui Zheng, Singapore (SG); Chengkuo Lee, Singapore (SG)

(73) Assignee: NATIONAL UNIVERSITY OF SINGAPORE, Singapore (SG)

(21) Appl. No.: 13/265,787

(22) PCT Filed: Apr. 23, 2010
(86) PCT No.: PCT/SG2010/000164

§ 371 Date: Dec. 30, 2011

Related U.S. Application Data
(60) Provisional application No. 61/171,913, filed on Apr. 23, 2009.

(51) Int. Cl. A61M 5/00; B23P 17/04; A61M 35/00; B82Y 5/00

(52) U.S. Cl. 604/506; 604/173; 29/428; 29/458; 29/592; 977/778

(57) ABSTRACT

An apparatus that includes a nano-projection array and an application unit configured to displace the nano-projection array to thereby deliver a composition (e.g., vaccine) to a controlled depth within the skin. The nano-projection array includes nano-projections carried by a carrier substrate. The application unit includes a peripheral structure and a displaceable carrier. The nano-projection array can be disposed within the peripheral structure. Displacement of the displaceable carrier causes a corresponding displacement to the nano-projection array. The displacement of the displaceable carrier can be controlled. For instance the distance of displacement of the displaceable carrier can be controlled. A method or process for manufacturing the apparatus is also provided.

### CROSS-REFERENCE TO RELATED APPLICATION

The present application is the U.S. national stage of international patent application number PCT/SG2010/000164, filed 23 Apr. 2010, and claims the benefit thereof under 35 U.S.C. §119(a) and 35 U.S.C. §365(a). The disclosure of that international application is hereby incorporated herein by reference.

### TECHNICAL FIELD

The present disclosure relates generally to apparatuses and methods for administering or delivering a biological, pharmaceutical, or chemical composition, for example a vaccine, into the body of an organism. More specifically, aspects of the present disclosure relate to systems, devices, and methods that involve nano-sized projections to administer or deliver a biological, pharmaceutical, or chemical composition to a controlled depth within the skin of an organism.

### BACKGROUND

There are biological, pharmaceutical, and chemical compositions (e.g., vaccines) available in the market for use in preventive inoculation and/or treatment against various diseases. Conventionally, the administration of vaccines into a body of an organism involves needle injections, for example subcutaneous or intramuscular injections. Other known methods for the administration of vaccines include oral, sublingual, or nasal applications.

The efficacy of administering vaccines via needle injections has been proven. However, there are several drawbacks in terms of safety and acceptability associated with such needle injections (1). A disadvantage or issue associated with the use of needle injections for delivering vaccines and other chemical compositions is a potential for opportunistic infections and transmission of blood borne diseases caused by viruses, for instance Hepatitis A, B, C and HIV viruses. Hence, in many cases, the use of needles for administering vaccines and/or other chemical compositions is associated with a prophylaxis with expensive and toxic drugs. An additional disadvantage of the use of needles is that many patients are needle-averse, which can lead to significant problems with compliance.

There are a few non-invasive delivery routes for vaccines and other chemical compositions, for example pulmonary, nasal, and oral delivery routes. Generally, nasal administration of vaccines and other chemical compositions offers a particularly ready access to a range of bodily systems, for example the body's systemic circulation, without the need to cross body barriers such as the Stratum Corneum, which hinders transdermal applications. However, the nasal administration or delivery of vaccines and other chemical compositions is still associated with several challenges or difficulties. Such challenges include ensuring an accurate quantification and/or sufficient residence time of an exact dose of a vaccine that is adsorbed either via specified cell(s) (e.g., M-cells) or by paracellular absorption through the nose. Other difficulties associated with the nasal delivery of vaccines and other chemical compositions include occurrence of unwanted deposition in the lungs and stomach as well as microbial contamination of multi-use devices that are used for effecting said nasal delivery (2).

Oral administration or delivery is not always recommended for vaccines since the continuous ingestion of food and the like substances has resulted in a high tolerance of the gastrointestinal tract. Accordingly, the effective immune response or immunity that can be generated via the oral administration route is typically significantly low. It addition, there are only a few known vaccines that can be well absorbed via the gastrointestinal tract, more specifically via the M-cells that line portions of the gastrointestinal tract. The relatively poor absorption of vaccines via the gastrointestinal tract is also due to the hostile environment both in the stomach (i.e., the acidic pH present in the stomach) and in the small intestine (i.e., the high enzyme concentration present in the small intestine).

The skin is the largest organ of the human body. The skin is highly immunogenic possessing a high concentration of dendritic cells, which are known as Langerhans cells. When primed by allergens, Langerhans cells migrate immediately to the lymph nodes for initiating corresponding immune responses. The skin, more specifically the outer layer of the skin or Stratum Corneum (SC), also has a protective function serving as a barrier for entry into the body of pathogens and other potentially harmful agents. However, the skin, more specifically the SC, simultaneously prevents therapeutic delivery of many drugs, particularly drugs of a molecular mass greater than approximately 1,000 daltons (Da).

Micrometer-scale microneedles have been used for effecting transdermal delivery of vaccines. For example, micrometer-scale microneedles that are capable of piercing animal and human cadaver skin for effecting transdermal delivery of small molecules such as proteins, DNA, and vaccine formulations for systemic action have been developed by Praunsnitz (3).

There are a number of limitations and disadvantages associated with the use of currently available micrometer-scale needles. Embodiments of the present disclosure provide improvements and/or alternative options to currently available options for the administration or delivery of biological, pharmaceutical, and chemical compositions.

### SUMMARY

Embodiments of the disclosure described herein provide systems, apparatuses, methods, processes, and techniques for administering or delivering biological, pharmaceutical, and chemical compositions, for example vaccines, drugs, therapeutic agents, and other biological or bioactive compounds, into a body of an organism. Many embodiments of the present disclosure provide systems, apparatuses, methods, processes, and techniques for administering or delivering biological, pharmaceutical, and chemical compositions to target sites within a body, for instance to the epidermis, of an organism with the use of nano-sized projections (or nano-projections), for instance nano-rods, nano-wires, or nano-needles, or nano-tubes.

In accordance with an embodiment of the present disclosure, there is disclosed an apparatus including a set of nano-sized projections carried by a carrier medium, the set of nano-sized projections shaped and configured to deliver a composition to a target site within a body. The apparatus further includes a peripheral structure configured to receive the set of nano-sized projections at least partially therewithin and a displaceable carrier coupled to the peripheral structure and configured to be displaceable relative to the peripheral structure between a first position and a second position. The displacement of the displaceable carrier relative to the peripheral structure facilitates or effectuates a corresponding displacement of the set of nano-sized projections for inserting the set of nano-sized projections into the body.

In accordance with another embodiment of the present disclosure, there is disclosed a system including at least two nano-sized projection arrays, each nano-sized projection array including a set of nano-sized projections carried by a carrier medium. The system also includes a set of peripheral structures configured to receive the at least two nano-sized projection arrays at least partially therewithin and a set of displaceable carriers coupled to the set of peripheral structures. The set of displaceable carriers is couplable to the at least two nano-sized projection arrays and configured to be displaceable between a first position and a second position relative to the peripheral structure for displacing the at least two nano-sized projection arrays coupled thereto.

In accordance with another embodiment of the present disclosure, there is disclosed a method for manufacturing an apparatus that is configured to deliver a composition to a target site within a body. The method includes forming a set of nano-sized projections supported by a carrier medium, the set of nano-sized projections shaped and configured for delivering the composition to the target site, and disposing the set of nano-sized projections at least partially within a peripheral structure. The method also includes coupling a displaceable carrier to the peripheral structure, the displaceable carrier couplable to the set of nano-sized projections and configured to be displaceable between a first position and a second position relative to the peripheral structure to thereby one of facilitate and effectuate a corresponding displacement of the set of nano-sized projections for inserting the set of nano-sized projections into the body.

In accordance with another embodiment of the present disclosure, there is disclosed a method including forming a plurality of segmented nano-sized projections supported by a carrier medium, wherein each of the plurality of segmented nano-sized projections includes at least two segments that are stacked relative to each other, and wherein at least a portion of the plurality of segmented nano-sized projections has a generally layered shape. The method also includes configuring the plurality of segmented nano-sized projections to be displaceable into a body for one of facilitating and effectuating delivery of a composition to a target site within the body.

In accordance with another embodiment of the present disclosure, there is disclosed a method of manufacturing a system for delivering at least one composition to a target site within a body. The method includes forming at least two nano-sized projection arrays, each nano-sized projection array including a set of nano-sized projections that is carried by a carrier medium, and disposing each nano-sized projection array within a peripheral structure. The method further includes coupling each nano-sized projection array to a displaceable carrier, the displaceable carrier configured to be displaceable between a first position and a second position relative to the peripheral structure to thereby one of facilitate and effectuate a corresponding displacement of the nano-sized projection array coupled thereto.

In accordance with further embodiment of the present disclosure, there is disclosed a method for transporting a composition to a target site. The method includes providing a nano-sized projection array, the nano-sized projection array including a set of nano-sized projections and a carrier medium carrying the set of nano-sized projections, the nano-sized projection array disposed at least partially within a peripheral structure and carried by a displaceable carrier. The method also includes displacing the displaceable carrier relative to the peripheral structure, wherein the displacement of the displaceable carrier structure relative to the peripheral structure facilitates or effectuates an application of force onto the nano-sized projection array carried by the displaceable structure to thereby displace the nano-sized projection array for transporting the composition to the target site. In accordance with yet another embodiment of the present disclosure, the method above can be for transporting a composition to a target site within a body. Accordingly, the displacement of the displaceable carrier structure relative to the peripheral structure one of facilitates and effectuates an application of force onto the nano-sized projection array to thereby displace the nano-sized projections of the nano-sized projection array for insertion into the body.

### BRIEF DESCRIPTION OF THE DRAWINGS

Embodiments of the present disclosure are described hereinafter with reference to the figures, in which:

FIG. 1A is a schematic representation of a nano-sized projection array for administering or delivering a pharmaceutical composition according to an embodiment of the present disclosure;

FIG. 1B is a schematic representation of another nano-sized projection array for administering or delivering a pharmaceutical composition according to an embodiment of the present disclosure;

FIG. 1C is a schematic representation of yet another nano-sized projection array for administering or delivering a pharmaceutical composition according to an embodiment of the present disclosure;

FIG. 1D is a schematic representation of a nano-sized projection array that includes nano-needles for administering or delivering a pharmaceutical composition according to another embodiment of the present disclosure;

FIG. 2A to FIG. 2D show partial side views of nano-sized projection arrays that include different numbers of segments;

FIG. 3A to FIG. 3D show partial top views of the nano-sized projection arrays of FIG. 2A to FIG. 2D respectively;

FIG. 4A is an isometric schematic representation of a nano-projection array including nano-projections carried by a silicon carrier substrate according to an embodiment of the present disclosure;

FIG. 4B is an isometric schematic representation of a nano-projection array including nano-projections carried by a PDMS carrier substrate according to an embodiment of the present disclosure;

FIG. 4C is an isometric schematic representation of a nano-projection array including nano-projections carried by a multi-layer carrier substrate that includes a PDMS layer and a silicon layer according to an embodiment of the present disclosure;

FIG. 5A is a schematic representation of an apparatus that includes a nano-projection array coupled to an application unit as according to an embodiment of the present disclosure;

FIG. 5B is a schematic top view of a nano-projection array that is disposed within a peripheral structure and carried on a displaceable carrier according to an embodiment of the present disclosure;

FIG. 6A is an isometric schematic representation of a nano-projection array that is disposed at an extended position according to an embodiment of the present disclosure;

FIG. 6B is an isometric schematic representation of the nano-projection array of FIG. 6B that is disposed at a retracted position according to an embodiment of the present disclosure;

FIG. 7A is a view of an apparatus including a nano-projection array that disposed within a peripheral structure and coupled to a displaceable carrier according to an embodiment of the present disclosure;

FIG. 7B is another view of the apparatus of FIG. 7A;

FIG. 8 shows an apparatus that includes a nano-projection array disposed within a peripheral structure of an application unit with a sealing film adhered to the peripheral structure in accordance with an embodiment of the present disclosure;

FIG. 9 is a schematic representation of an apparatus including a nano-projection array and an application unit that includes a displacement control element according to an embodiment of the present disclosure;

FIG. 10 is a schematic representation of an apparatus including a nano-projection array and an application unit that includes a displacement control element and a set of force management elements according to an embodiment of the present disclosure;

FIG. 11A is a schematic representation of a nano-projection array carried by a displaceable carrier, the nano-projection array and displaceable carrier being disposed at a retracted position;

FIG. 11B is a schematic representation of the nano-projection array carried by the displaceable carrier of FIG. 11A, the nano-projection array and displaceable carrier being disposed at an extended position;

FIG. 12A shows a nano-projection array coupled to a set of force management elements in accordance with an embodiment of the present disclosure;

FIG. 12B shows a nano-projection array coupled to a different set of force management elements in accordance with an embodiment of the present disclosure;

FIG. 13 is a schematic representation of an apparatus including a nano-projection array and an application unit that includes a base substrate with a fluidic reservoir formed therein according to an embodiment of the present disclosure;

FIG. 14 is a schematic representation of a system including multiple nano-projection arrays in accordance with an embodiment of the present disclosure;

FIG. 15A is a view of a system that includes multiple nano-needle arrays with a number of application units that include a fluidic reservoir in accordance with an embodiment of the present disclosure;

FIG. 15B is a top view of the system of FIG. 14A;

FIG. 16 is a flowchart of a process for manufacturing an apparatus for delivering a composition according to an embodiment of the present disclosure;

FIG. 17 is a flowchart of a process for manufacturing a system for delivering at least one composition according to an embodiment of the present disclosure;

FIG. 18A is a first view of a nano-needle array that includes nano-projections carried by a silicon carrier substrate formed during a process of manufacturing a nano-needle array according to an embodiment of the present disclosure;

FIG. 18B is a second view of the nano-needle array of FIG. 18A;

FIG. 19A is a first view of a nano-needle array that includes nano-projections carried by a carrier substrate with a silicon layer and a PDMS layer as formed during a process of manufacturing a nano-needle array according to an embodiment of the present disclosure;

FIG. 19B is a second view of the nano-needle array of FIG. 19A;

FIG. 20A is a first view of a nano-needle array that includes nano-projections carried by a PDMS carrier substrate as formed during a process of manufacturing a nano-needle array according to an embodiment of the present disclosure;

FIG. 20B is a second view of the nano-needle array of FIG. 20A;

FIG. 21A shows a partial isometric view of nano-projections in a state prior to skin penetration or insertion as obtained using scanning electron microscopy;

FIG. 21B shows a partial isometric view of the nano-projections of FIG. 17A in a state after skin penetration or insertion as obtained using scanning electron microscopy;

FIG. 22A shows a top view of Albumin-FITC distribution of a nano-projection array of an apparatus in accordance with an embodiment of the present disclosure;

FIG. 22B is an enlarged view of the Albumin-FITC distribution shown in FIG. 18A;

FIG. 23 shows a top view (A) and side views (B) and (C) of a skin sample after insertion of nano-projections into a skin sample, wherein fluorescent channels formed by the penetration of Albumin-FITC into the skin sample are visible;

FIG. 24 is a representative three-dimensional structure of a tape stripping, which shows fluorescence all along the depth of a skin sample;

FIG. 25 represents the working principle of the VapoMeter used in association with experiments conducted in association with particular embodiments of the present disclosure;

FIG. 26 is a graph show representative results of experiments evaluating trans-epidermal water loss (TEWL) as performed in association with particular embodiments of the present disclosure; and

FIG. 27 is a graphical representation of the titers of IgG as measured or obtained in mice after 5 weeks from the insertion of nano-projections into the skin of the mice in association with particular embodiments of the present disclosure.

### DETAILED DESCRIPTION

There exist several conventional methods and devices for administering pharmaceutical compositions, for example drugs, vaccines, and other biologically active molecules, into a body. For instance, needle injections have been conventionally used to administer or deliver vaccines into a body. However, there are limitations and disadvantages associated with the use of existing needles for administering or delivering pharmaceutical compositions into a body. Some of such limitations and disadvantages include patients' fear of needles, infliction of pain, and potential risk for needle contamination and consequent cross-infection.

Embodiments of the present disclosure relate to systems, apparatuses, devices, methods, processes, and techniques for administering, delivering, providing, or transporting biological, pharmaceutical, and other chemical compositions, into a body. More specifically, most embodiments of the present disclosure relate to use of nano-sized structures or projections, for example nano-sized needles (or nano-needles) and/or nano-sized rods (or nano-rods), for administering, delivering, providing, or transporting biological, pharmaceutical, and other chemical compositions to a predictable, generally predictable, controlled, or generally controlled depth or position within a target structure, tissue, or site in a body, such as the epidermis. Many embodiments of the present disclosure address at least one limitation, disadvantage, or issue associated with existing methods and/or devices for administering or delivering pharmaceutical compositions such as vaccines into a body.

For purposes of the present disclosure, biological, pharmaceutical, or chemical compositions can be understood to include vaccines, drugs, and other bioactive or bio-therapeutic molecules, agents, formulations, or compositions, which are capable of providing a protective, immunomodulatory, immunogenic, and/or therapeutic effect when administered or delivered into the body of a living organism. Bioactive or bio-therapeutic molecules, agents, formulations, or compositions can include polynucleotides, nucleic acids, antigens, allergens, adjuvants, polypeptides, anti-oxidants, anti-cancer, anti-mutagenic, anti-neoplastic, and/or other like compounds or biological molecules. In addition, biological or pharmaceutical compositions can include formulations or compositions that are specifically designed or formulated to optimize the performance of a composition or substance within a body, for instance to induce an enhanced or optimized protective and/or therapeutic efficacy. Optimization or formulation can include adjusting the concentration of active ingredient(s) as well as inclusion of stabilizing agents(s), solvent(s), and/or a like compound(s). In certain embodiments, a composition for delivery to a target site can include nano-particles.

In addition, the nano-sized projections of the present disclosure can include, or can be, nanorods, nanowires, nanoneedles, nano-tubes, and like structures having sizes in the nano-meter range. More particularly, a diameter or cross-sectional area of portions of the nano-sized projections that penetrate skin or body tissue are in the nano-meter range. In most embodiments of the present disclosure, a target site refers to a site within the skin, and more specifically to a site within the epidermis of the skin.

The systems, apparatuses, methods, and processes of the present disclosure utilize nano-sized projections that are sized, shaped, and/or configured to facilitate or effectuate administration of a biological, pharmaceutical, or chemical composition to a target site within a body. In most embodiments, the nano-sized projections are sized, shaped, and/or configured to be displaced to, and be positioned at, the target site. The displacement, and position, of the nano-sized projections at the target site facilitate or effectuate the administration or delivery of the biological, pharmaceutical, or chemical composition to the target site.

In many embodiments, the administration or delivery of the biological, pharmaceutical, or chemical composition to the target site is effective for providing, producing, causing, generating, or inducing a biological response within the body. The biological response is for example a therapeutic, protective, immunogenic, and/or immuno-modulatory response.

In embodiments of the present disclosure, the displacement and insertion of the nano-sized projections into the body, for instance to the target site, can be controlled or managed. In many embodiments, the distance at which each nano-sized projection is inserted into the body can be controlled, for example selected and/or varied. For instance, in several embodiments, displacement of the nano-sized projections is controlled for preferentially targeting a tissue or skin layer (e.g. the epidermis) or cells of a tissue of skin layer (e.g., Langerhans cells). In addition, the displacement of the nano-sized projections can be controlled for specifically avoiding contact with a tissue or skin layer (e.g., the dermis) or particular types of cells within a tissue or skin layer (e.g., sensory nerve ending). In numerous embodiments, the nano-sized projections can be inserted or injected into the body at a uniform and/or consistent pressure.

Apparatuses or devices of most embodiments of the present disclosure include an application unit that is configured for facilitating or effectuating displacement of the nano-projections that are coupled thereto, or carried thereby.

In most embodiments, the application unit includes a peripheral housing (also known as a peripheral structure or box). The nano-sized projections can be housed or disposed at least partially within the peripheral housing. In particular embodiments, the peripheral housing is configured to surround or isolate the nano-sized projections that are disposed therewithin to thereby protect the nano-sized projections.

In most embodiments, the application unit includes a displaceable carrier or a displaceable structure. The displaceable carrier can be displaced relative to the peripheral structure. The displacement of the displaceable carrier relative to the peripheral structure facilitates or effectuates a displacement of the nano-sized projections. In numerous embodiments, the displaceable carrier can be referred to as a force transfer structure or element that is configured to transfer a force to the nano-sized projections for inserting the nano-sized projections into the body.

In many embodiments, the application unit is configured to control or manage the displacement and insertion of the nano-sized projections into the body. In numerous embodiments, the application unit includes a displacement control element. The displacement control element is disposed and/or configured to control displacement of the displaceable carrier and the nano-projections. In several embodiments, the displacement control element is disposed and/or configured to control a distance of displacement of the displaceable carrier, and therefore a distance of displacement of the nano-projections.

In certain embodiments, the displacement control element is instead, or includes, a force transfer element. The force transfer element facilitates or effectuates transfer of a force that is applied thereto onto the displaceable carrier.

In some embodiments of the present disclosure, a set of force management elements (also known as force distribution elements or force distributors) can be used to aid the control or management of the displacement of the nano-sized projections, and therefore the insertion of the nano-sized projections into the body. For instance, in particular embodiments, the set of force management elements are configured to control a force or pressure that is applied, or transferred, to the displaceable carrier, the nano-sized projection array, and/or the nano-sized projections of the nano-sized projection array.

In some embodiments, the set of force management elements is configured to distribute (e.g., evenly distribute) and/or limit the force or pressure that is applied, or transferred, to the displaceable carrier, the nano-sized projection array, and/or the nano-sized projections of the nano-sized projection array. The control, distribution, or limitation of the force or pressure applied, or transferred, to the nano-sized projections can help to facilitate or effectuate the insertion of the nano-sized projections into the body at a uniform pressure and/or depth.

### Structural Aspects of Particular Embodiments

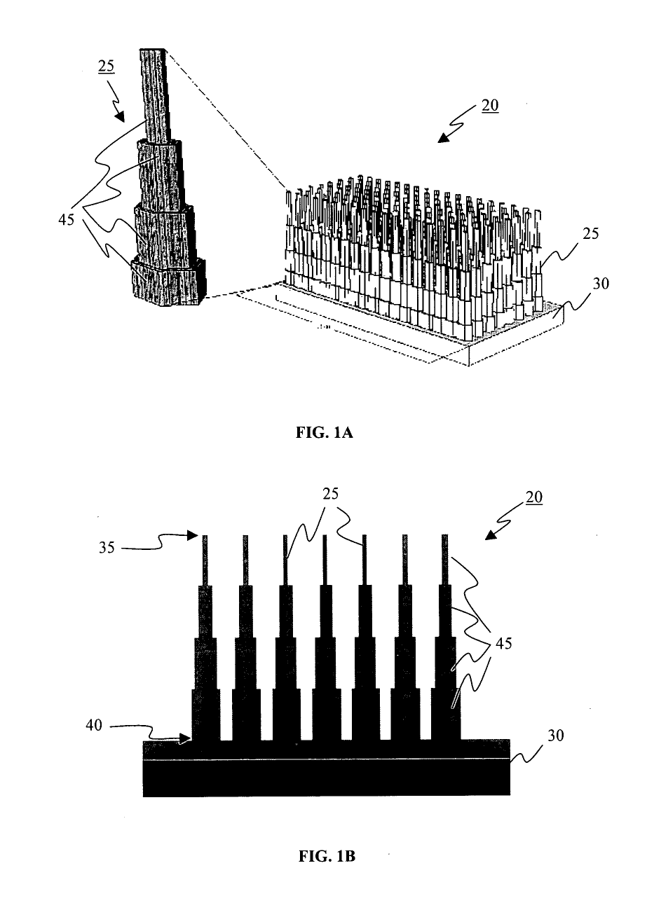

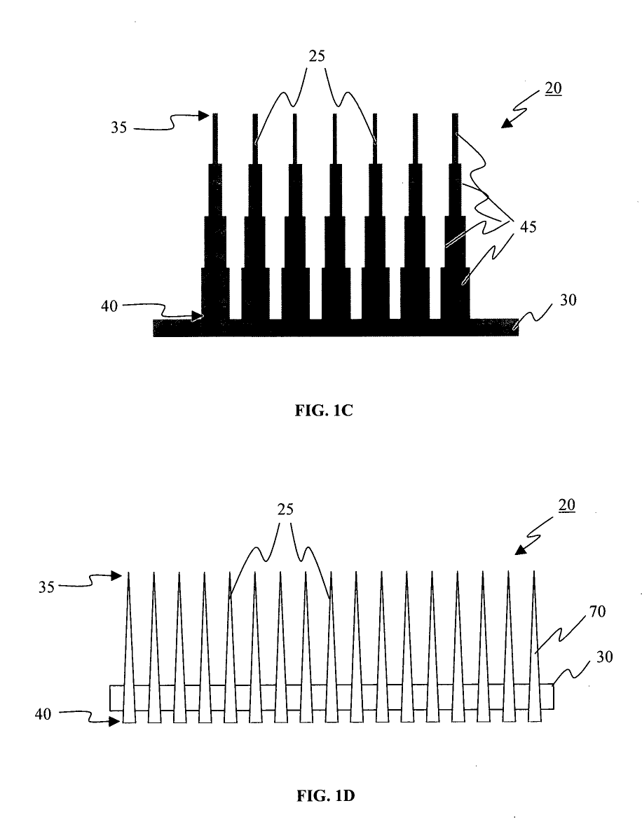

FIG. 1A to FIG. 15B show structural aspects of various embodiments of the present disclosure.

Systems, apparatuses, and devices of the present disclosure include nano-sized projections (hereinafter referred to as nano-projections), which are shaped, dimensioned, and/or configured to be inserted, injected, or displaced into a body of an organism. The nano-projections are shaped, dimensioned, and/or configured to be inserted to a target site within the body, for instance the epidermis of the skin. In numerous embodiments, the insertion of the nano-projections to the target site within the body facilitates or effectuates administering or delivering of a biological, pharmaceutical, or chemical composition (hereinafter referred to as a composition), for example a vaccine, to the target site to thereby induce, provide, generate, or produce a therapeutic, protective, immunogenic, and/or immuno-modulatory effect.

### Structural Aspects of Particular Nano-Projection Arrays

FIG. 1A to FIG. 1D show structural aspects of particular nano-projection arrays 20 in accordance with various embodiments of the present disclosure. Each nano-projection array 20 can be alternatively referred to as a set of nano-projections or an array of nano-projections.

In most embodiments, each nano-projection array 20 includes a number of nano-projections 25 (or a set of nano-projections 25) and a carrier substrate 30 (also known as a carrier medium, support substrate, support medium, base substrate, or base medium). Apparatuses, devices, and systems provided by embodiments of the present disclosure include at least one, and in some embodiments two, three, four, ten, or more, nano-projection arrays 20.

In most embodiments, the nano-projection array 20 includes at least approximately 500 nano-projections 25. In many embodiments, the nano-projection array 20 includes at least approximately 3,600 nano-projections 25. In numerous embodiments, the nano-projection array 20 includes at least approximately 10,000 nano-projections 25. In several embodiments, the nano-projection array 20 includes at least approximately 100,000 nano-projections 25, for instance at least approximately 500000, 1 million, 5 million, 9 million, or even more nano-projections 25.

The nano-projections 25 are shaped, dimensioned, and/or configured for insertion, injection, or displacement into a body. The nano-projections 25 are also shaped, dimensioned, and/or configured to facilitate or effectuate delivery of a composition (e.g., a vaccine) to the target site within the body.

In many embodiments of the present disclosure, for example as shown in FIG. 1A to FIG. 1D, a nano-projection 25 has a tapered, conical, layered, stacked, and/or segmented shape. Accordingly, in many embodiments, a nano-projection 25 has a first end 35 (or first tip 35) with a smaller diameter as compared to a second end 40 (or second tip 40), each of the first and second ends 35, 40 being on opposite ends of the nano-projection 25.

The shape of a nano-projection 25 can facilitate insertion of the nano-projection 25 into the body. In various embodiments, the tapered, conical, layered, stacked, and/or segmented shape of a nano-projection 25 provides an enhanced structural integrity, or strength, for the nano-projection 25. In particular embodiments, the tapered, conical, layered, stacked, and/or segmented shape of a nano-projection 25 aids a controlled or managed insertion of the nano-projection 25 into the body.

In many embodiments, the diameter of the first end 35 of a nano-projection 25 is at least approximately 10% smaller than the diameter of the second end 40 of the nano-projection 25. In several embodiments, the diameter of the first end 35 of a nano-projection 25 is at least approximately 20% smaller than the diameter of the second end of the nano-projection 40. In various embodiments, the diameter of the first end 35 of a nano-projection 25 is at least approximately 40%, for example approximately 50%, 60%, or 75% smaller than the diameter of the second end 40 of that nano-projection 25.

In numerous embodiments, the diameter of the first end 35 of a nano-projection 25 is between approximately 10 nm and 250 nm. In several embodiments, the diameter of the first end 35 of a nano-projection 25 is between approximately 20 nm and 200 nm. In various embodiments, the diameter of the first end 35 of a nano-projection 25 is between approximately 25 nm and 100 nm. In particular embodiments, the diameter of the first end 35 of a nano-projection 25 is approximately 25 nm, 30 nm, 35 nm, 40 nm, 45 nm, 50 nm, 60 nm, 70 nm, 80 nm or 90 nm.

In numerous embodiments, the diameter of the second end 40 of a nano-projection 25 is between approximately 50 nm and 400 nm. In several embodiments, the diameter of the second end 40 of a nano-projection 25 is between approximately 100 nm and 300 nm. In various embodiments, the diameter of the second end 40 of a nano-projection 25 is between approximately 125 nm and 200 nm. In particular embodiments, the diameter of the second end 40 of a nano-projection 25 is approximately 140 nm, 150 nm, 160 nm, or 170 nm.

Although tapered, conical, layered, or segmented shaped nano-projections 25 are described in the present disclosure, one or more sets of nano-projections 25 having alternative shapes, sizes, and/or configurations are encompassed by the scope of the present disclosure. For example, at least one set of nano-projections 25 can be cylindrical or rectangular.

In most embodiments of the present disclosure, the length of each nano-projection 25 can be selected and varied, for instance depending upon the target site for delivery of the composition (e.g., vaccine). In many embodiments, the nano-projections 25 have a length configured to preferentially target a particular skin layer, for example the epidermis, for delivering the composition (e.g., vaccine) to the particular skin layer. In numerous embodiments, the length of the nano-projections 25 is selected to enable preferential insertion to the epidermis, while specifically avoiding other body tissue (e.g., the dermis).

Although preferential insertion of the nano-projections 25 to the epidermis, and specific avoidance of contact between the nano-projections 25 and the dermis, is described above, selection of an alternative length of the nano-projections 25 is also included within the scope of the present disclosure. For instance, selection of the length of the nano-projections 25 for preferential insertion through the epidermis and into the dermis, while avoiding other body tissues located at a deeper depth within the body, is also within the scope of the present disclosure.

### Topography of the Skin

The skin commonly refers to the outer covering of an organism. Mammalian skin is generally composed of three main layers, namely the epidermis, the dermis, and the hypodermis. The epidermis is the outermost layer of the skin and forms a substantially waterproof and protective cover over the surface of the body. The epidermis has no blood vessels. The epidermis includes Merkel cells, keratinocytes, melanocytes, and Langerhans cells. Langerhans cells in the epidermis are dendritic cells that are part of the adaptive immune system. In the present disclosure, Langerhans cells are capable of functioning as antigen-presenting cells that facilitate generation, inducement, or building, of an immune protection (or immunity).

The dermis is the layer of the skin beneath the epidermis. The dermis includes hair follicles, sweat glands, sebaceous glands, apocrine glands, lymphatic vessels, and blood vessels. The hypodermis is disposed below the dermis and functions to connect the skin, more specifically the epidermis and the dermis, to underlying bone and muscle tissue. The hypodermis includes loose connective tissue and elastin. The main cell types of the hypodermis include fibroblasts, macrophages, and adipocytes (also known as fat-storage cells). The hypodermis can also be known as the subcutaneous tissue.

In many embodiments of the present disclosure, the length of the nano-projections 25 correlates to a depth of a target site, tissue, or skin layer within the body. In most embodiments, the length of the nano-projections 25 is between approximately 5 μm and 200 μm. In many embodiments, the length of the nano-projections 25 is between approximately is between approximately 10 μm and 150 μm. In several embodiments, length of the nano-projections 25 is between approximately 20 μm and 100 μm. In particular embodiments, the length of the nano-projections 25 is approximately 25 μm, 40 μm, 50 μm, 60 μm, or 75 μm.

In several embodiments, the transport or delivery of the composition (e.g., vaccine) to the epidermis provides, or presents, the Langerhans cells that are located at the epidermis with the composition. In various embodiments, the presentation of the composition to the Langerhans cells located within the epidermis facilitates or causes an inducement, generation, or production of a therapeutic, protective, immunogenic, or immunomodulatory response within the body. For example, in particular embodiments, where the composition is a vaccine, the delivery of the vaccine to the epidermis (and Langerhans cells located thereat) facilitates an inducement of an associated immune response (or immunity) within the body.

The length of the nano-projections 25 of several embodiments of the present disclosure, and the managed or controlled insertion thereof into the body, enable management, reduction, or elimination of pain that is conventionally associated with needle injections. This is because the nano-projections 25 of numerous embodiments of the present disclosure are of a length only sufficient to be preferentially inserted to (i.e., to reach) the epidermis of the skin, and not the dermis of the skin where the body's sensatory tissue (i.e., sensatory nerve endings) is located. As the nano-projections 25 of numerous embodiments of the present disclosure do not reach, and contact, the sensatory nerve endings located at the dermis, the management, reduction, or elimination of pain can be achieved.

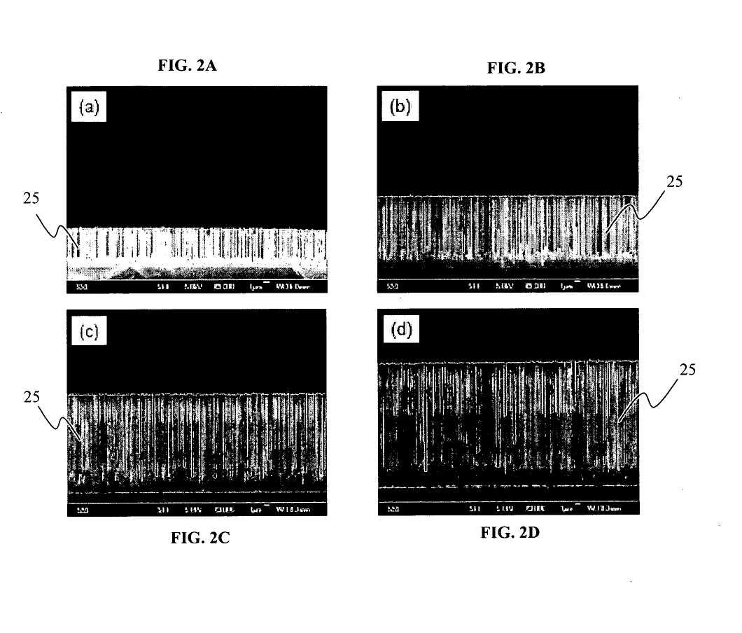

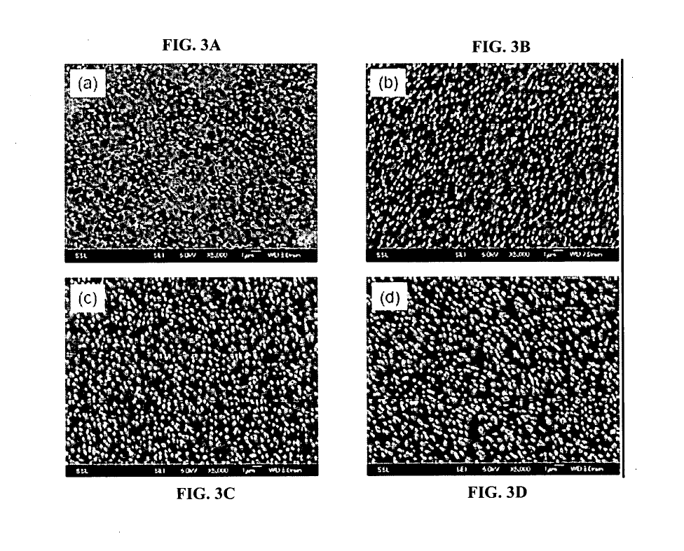

FIG. 2A to FIG. 2D show different nano-projection arrays 20 that include nano-projections 25 with different numbers of segments or portions 45. FIG. 3A to FIG. 3D show are top views of the nano-projection arrays 20 of FIG. 2A to FIG. 2D respectively.

In various embodiments of the present disclosure, the nano-projections 25 can include a number of segments 45 or portions (i.e., the nano-projection 25 can be segmented). For example, in particular embodiments, the nano-projections 25 include at least two segments 45. In certain embodiments, the nano-projections 25 include at least three segments 45, for example three, four, five, or more segments 45. In certain embodiments, the segmented nano-projections 25 can exhibit an enhanced structural strength or integrity relative to non-segmented nano-projections.

The length of each segment 45 can be selected and varied, for instance depending upon total length of the nano-projection 25, target tissue type or skin layer, and/or type of pharmaceutical composition to be administered by the apparatus. For instance, in particular embodiments, the length of each segment 45 can be between approximately 1 μm and 50 μm, and more particularly approximately 2 μm, 5 μm, 10 μm, 15 μm, 20 μm, or 25 μm.

### Structural Aspects of a Carrier Substrate or Medium 30

In the embodiments of the present disclosure, the nano-projection array 20 includes a carrier substrate 30 or carrier medium 30. The carrier substrate 30 is shaped, sized, and/or configured for carrying, holding, and/or supporting the set of nano-projections 25 of the nano-projection array 20.

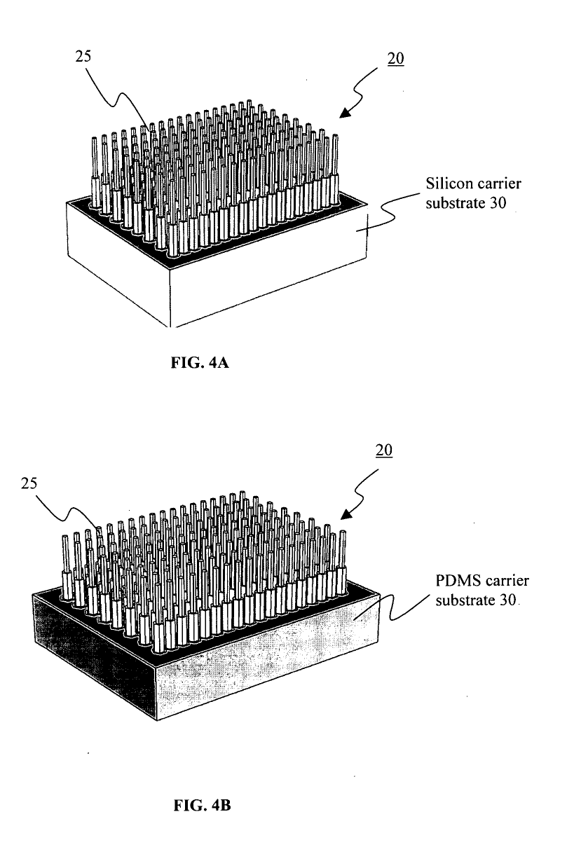

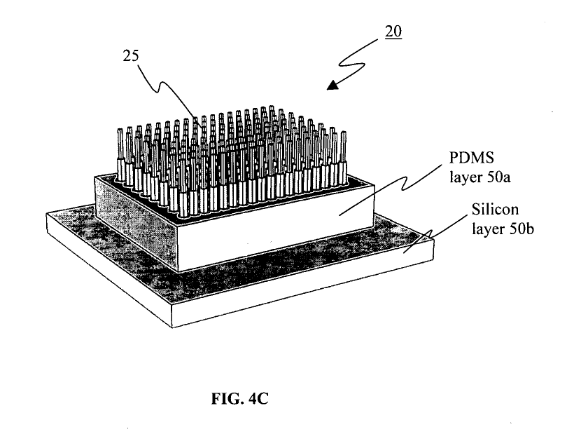

FIG. 4A to FIG. 4C, as well as FIG. 1A to FIG. 1D, shows carrier substrates 30 according to particular embodiments of the present disclosure. In many embodiments, the carrier substrate 30 is shaped, sized, and/or configured to hold, set, or maintain the set of nano-projections 25 as an integral unit, with a fixed or predictable spatial position and/or configuration relative to each other. In many embodiments, the carrier substrate 30 is planar, or substantially planar. The set of nano-projections 25 can extend or project from, or through, the planar carrier substrate 30 at a perpendicular, or substantially perpendicular, angle to a surface, or a plane, of the planar carrier substrate 30.

The size, thickness, shape, and/or configuration of the carrier substrate 30 can be selected and varied, for instance depending upon a number of nano-projections 25 carried thereby, a configuration of the nano-projections 25 carried thereby, and/or a type of composition to be administered by the nano-projections 25. For instance, the shape of the carrier substrate 30 can be a square, rectangular, circular, triangular, or an irregular shape.

In many embodiments of the present disclosure, the carrier substrate 30 has a surface area of between approximately 5 mm² and 400 mm². In numerous embodiments, the carrier substrate 30 has a surface area of between approximately 20 mm² and 200 mm². In various embodiments, the carrier substrate 30 has a surface area of between approximately 25 mm² and 100 mm², for example approximately 40 mm², 50 mm², 60 mm², and 70 mm².

In many embodiments, the carrier substrate 30 has a thickness of between approximately 0.2 μm and 10 μm. In several embodiments, the carrier substrate 30 has a thickness of between approximately 0.5 μm and 7.5 μm. In various embodiments, the carrier substrate 30 has a thickness of between approximately 0.6 μm and 5 μm, for example approximately 0.8 μm, 1.0 μm, 1.5 μm, 2 μm, or 2.5 μm.

In many embodiments of the present disclosure, the number or the density of the nano-projections 25 carried by each carrier substrate 30 can be selected and varied, for instance depending upon type or dose of pharmaceutical composition delivered by the apparatus, and/or length of the nano-projection(s) 25. In many embodiments, the density of nano-projections 25 carried by a carrier substrate 30 is between approximately 10 per mm² and 500 per mm². In particular embodiments, the density of nano-projections 25 carried by a carrier substrate 30 is approximately 100 per mm², 250 per mm², or 400 per mm². In specific embodiments the density of nano-projections 25 carried by a carrier substrate 30 is more than 500 per mm², for instance least approximately 5,000 per mm².

In many embodiments of the present disclosure, a distance between adjacent or neighboring first ends 35 of the nano-projections 25 carried by the carrier substrate 30 is between approximately 0.1 μm and 1.50 μm. In some embodiments, the distance between adjacent or neighboring first ends 35 of the nano-projections 25 carried by the carrier substrate 30 is between approximately 0.25 μm and 1.0 μm. In various embodiments, the distance between adjacent or neighboring first ends 35 of the nano-projections 25 carried by the carrier substrate 30 is between approximately 0.4 μm and 0.4 μm for example approximately 0.5 μm, 0.6 μm, or 0.7 μm.

In many embodiments, the distance between adjacent or neighboring second ends 40 of the nano-projections 25 carried by the carrier substrate 30 is between approximately 0.05 μm and 0.75 μm. In several embodiments, the distance between adjacent or neighboring second ends 40 of the nano-projections 25 carried by the carrier substrate 30 is between approximately 0.1 μm and 0.5 μm. In various embodiments, the distance between adjacent or neighboring second ends 40 of the nano-projections 25 carried by the carrier substrate 30 is between approximately 0.2 μm and 0.4 μm, for example approximately 0.25 μm, 0.3 μm, or 0.35 μm.

In some embodiments, the carrier substrate 30 is a single layer structure. FIG. 1A, FIG. 1C, FIG. 1D, FIG. 4A, and FIG. 4B show carrier substrates 30 that include a single layer.

In particular embodiments, for instance as shown in FIG. 4A, the carrier substrate 30 is made at least substantially of silicon (Si). In other embodiments, for instance as shown in FIG. 4B, the carrier substrate 30 is made at least substantially of Polydimethylsiloxane (PDMS). The carrier substrate 30 can be alternatively made of, or substantially of, other materials, for instance other biocompatible or biodegradable material(s), within the scope of the present disclosure.

In certain embodiments, the carrier substrate 30 can include at least two layers 50, for example two, three, four, or more layers 50, which are coupled together. For example, FIG. 1B shows a carrier substrate 30 that includes two layers 50, namely a first layer 50a and a second layer 50b. Similarly, FIG. 4C shows a carrier substrate 30 that includes two layers 50, namely a first layer 50a made at least substantially of PDMS and a second layer 50b made at least substantially of Si.

In several embodiments wherein the carrier substrate 30 includes at least two layers 50, the size (e.g., surface area) and/or thickness of individual layers 50 can be identical, similar, distinct, or dissimilar with respect to each other. For instance, in particular embodiments, the first layer 50a can have a smaller surface area, for example a smaller surface area of approximately 10%, 20%, 25%, or more, as compared to the second layer 50b. In certain embodiments, the first layer 50a can be thinner, for example thinner by approximately 10, 20%, 40%, 50%, or more, as compared to the second layer 50b.

In some embodiments of the present disclosure, one layer 50 (e.g., the first layer 50a) of the carrier substrate 30 can be referred to as an interface layer or a seed layer. The interface layer can function as a supporting or stabilizing layer for the nano-projections. In addition, in particular embodiments, the interface layer can function as a platform or base for facilitating fabrication or manufacture of the nano-projections 25.

The insertion, injection, or displacement of the nano-projections 25 to a target site within body, for instance through the stratum corneum (SC) layer to the epidermis of the skin, facilitates or effectuates the administration or delivery of the composition (e.g., a vaccine) to the target site.

As further detailed below, the carrier substrate 30 is displaced to facilitate or effectuate the insertion, injection, or displacement of the nano-projections 25 that are carried thereby into the body. In embodiments of the present disclosure, the distance or depth of insertion of the nano-projections 25 into the body is controlled. In many embodiments, the displacement of the carrier substrate 30, for instance, distance of displacement of the carrier substrate 30, can be controlled or managed.

A distance of displacement of the carrier substrate 30, and hence nano-projections 25 carried thereby, can be controlled or managed, for instance selected and varied. Controlling the distance of displacement of the carrier substrate 30 facilitates or effectuates control of the distance of displacement of the nano-projections 25, and hence the distance of insertion of the nano-projections 25 into the body. In several embodiments, the distance of displacement of the carrier substrate 30 can be controlled for enabling the nano-projections 25 to preferentially reach the epidermis, while avoiding another skin layer or body tissue, for example the dermis. Alternatively, the distance of displacement of the carrier substrate 30 can be controlled for enabling the nano-projections 25 to preferentially reach the dermis, while avoiding other body tissues located at a deeper depth within the body.

### Structural Aspects of an Application Unit 100

In most embodiments of the present disclosure, an application unit 100 (also known as an applicator) facilitates, manages, or controls the displacement of the nano-projection array 20, more specifically the nano-projections 25 and the carrier substrate 30 of the nano-projection array 20, such that the nano-projections 25 can reach an intended target depth in the body (e.g., within the epidermis or dermis).

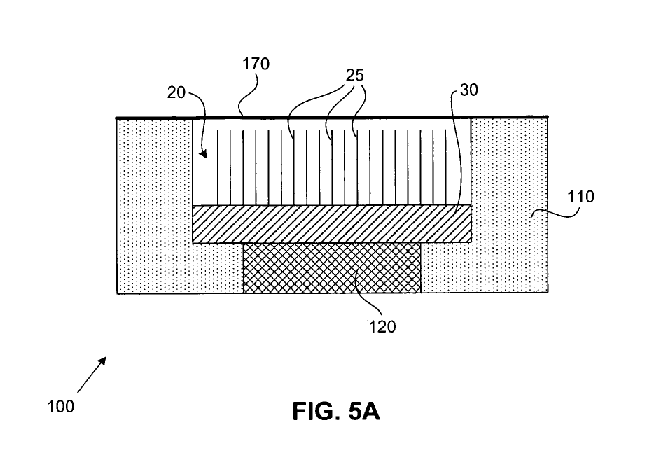

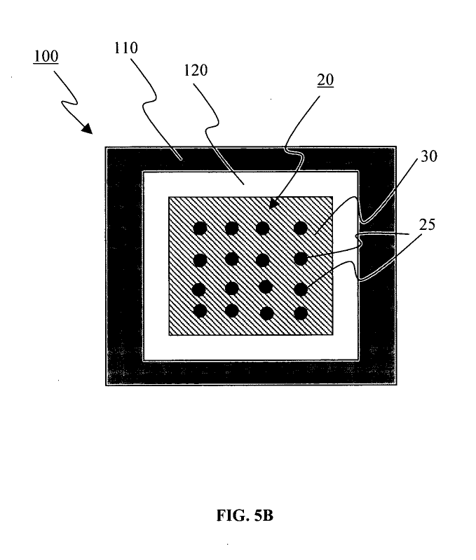

FIG. 5 to FIG. 15B show aspects of an application unit 100 according to particular embodiments of the present disclosure. In most embodiments, each application unit 100 carries, is coupled to, or is associated with, a nano-projection array 20.

### Structural Aspects of a Peripheral Structure or Peripheral Housing

In most embodiments of the present disclosure, the application unit 100 includes a peripheral structure 110 (also known as a peripheral support or a peripheral frame).

The peripheral structure 110 is shaped, dimensioned, and/or configured such that the nano-projection array 20 can be disposed or accommodated within, or substantially within, the peripheral structure 110. In other words, in numerous embodiments, the peripheral structure 110 is shaped, dimensioned, and/or configured to at least partially surround and/or isolate the nano-projection array 20, and therefore the nano-projections 25.

The breath and width of the peripheral structure 110 can be selected and varied, for instance depending upon the size of the nano-projection array 20, such as based upon a surface area of the carrier substrate 30. For example, each of the breath and width of the peripheral structure 110 can be between approximately 5 mm and 2.5 cm. In specific embodiments, the breath and width of the peripheral structure 110 can be between approximately 1 cm and 2 cm, for instance approximately 1.2 cm, 1.4 cm, or 1.6 cm.

A height of the peripheral structure 110 can depend upon the length of the nano-projections 25 and/or depth of the target site to which the composition is to be delivered. In various embodiments, the height of the peripheral structure 110 (i.e., a distance between a base surface and a top surface of the peripheral structure 110) can influence or determine a distance of displacement of the nano-projection array 20, and accordingly a distance that the nano-projections 25 extend beyond the top surface of the peripheral structure 110.

In some embodiments, the height of the peripheral structure 110 is between approximately 10 μm and 250 μm. In various embodiments, the height of the peripheral structure 105 is between approximately 25 μm and 150 μm. In particular embodiments, the height of the peripheral structure 105 is between approximately 50 μm and 125 μm, for example approximately 70 μm, 80 μm, 90 μm, or 100 μm.

In addition, a thickness of peripheral structure 110 can be selected and varied, for instance depending upon particular structural or functional characteristics of the application unit 100.

### Structural Aspects of a Displaceable Carrier/Base/Substrate

In many embodiments of the present disclosure, the application unit 100 further includes a displaceable carrier 120 or a displaceable base 120. The displaceable carrier 120 can also be referred to as a displaceable substrate, a movable frame, a movable carrier, a movable base, a movable substrate, or a like reference term.

The displaceable carrier 120 is configured to be displaceable relative to the peripheral structure 110 to displace the nano-projection array 20 that is disposed within the peripheral structure 110. In other words, in many embodiments, the displacement of the displaceable carrier 120 results in a corresponding displacement of the nano-projection array 20, and hence nano-projections 25 of the nano-projection array 20. In several embodiments, the displacement of the displaceable carrier 120 results in an application of force onto the nano-projection array 20 coupled to, carried by, or associated with the displaceable carrier 120.

In general, the displaceable carrier 120 is couplable to the nano-projection array 20. The position of the displaceable carrier 120 relative to the nano-projection array 20 can be determined and/or varied, for instance depending upon particular structural or functional characteristics of the present disclosure.

In many embodiments, the displaceable carrier 120 is positioned at, or proximal to, a bottom side (i.e., a base) of the carrier substrate 30 of the nano-projection array 20. In such embodiments, the nano-projection array 20 can be mounted onto, or carried by, the displaceable carrier 120.

FIG. 5B shows a nano-projection array 20 that disposed within a peripheral structure 110 and mounted onto a displaceable carrier 120. The displaceable carrier 120 of FIG. 5B can be a PDMS or silicon displaceable carrier 120 that is configured to allow transfer of a force applied thereto onto the nano-projection array 20 to thereby facilitate or effectuate displacement of the nano-projection array 20 and insertion of the nano-projections 25 of the nano-projection array 20 into the body.

In particular embodiments, the displaceable carrier 120 is positioned about, or at least somewhat about, the perimeter of the carrier substrate 30 (e.g., the displaceable carrier 120 is positioned around the nano-projection array 20). In some embodiments of the present disclosure, the displaceable carrier 120 is disposed at within, or least partially within, the peripheral structure 110 of the application unit 100.

As mentioned above, the displaceable carrier 120 can be displaced relative to the peripheral structure 110. In many embodiments, the displaceable carrier 120 can be displaced from, or between, a first position (also known as a retracted position or rest position) to, or toward, a second position (also known as an extended position or activated position) relative to the peripheral structure 110.

In most embodiments, the displacement of the displaceable carrier 120 between the first and second position (i.e., between the retracted and extended position) correspondingly displaces the nano-projection array 20 that is coupled to, carried by, or associated with the displaceable carrier 120 between a first (or retracted) and a second (or extended) position, respectively.

In many embodiments, the nano-projection array 20, is positioned within, or substantially within, the peripheral structure 110 when at the first (or retracted) position. Accordingly, when the nano-projection array 20 is at the first (or retracted) position, the nano-projections 25 thereof do not extend beyond a plane of the top surface of the peripheral structure 110. The nano-projection array 20 is positioned exterior, or at least partially external to, the peripheral structure 110 when disposed at or displaced to the second (or extended) position. Accordingly, when the nano-projection array 20 is at the second (or extended) position, the nano-projections 25 thereof extend at least partially beyond a plane of the top surface of the peripheral structure 110.

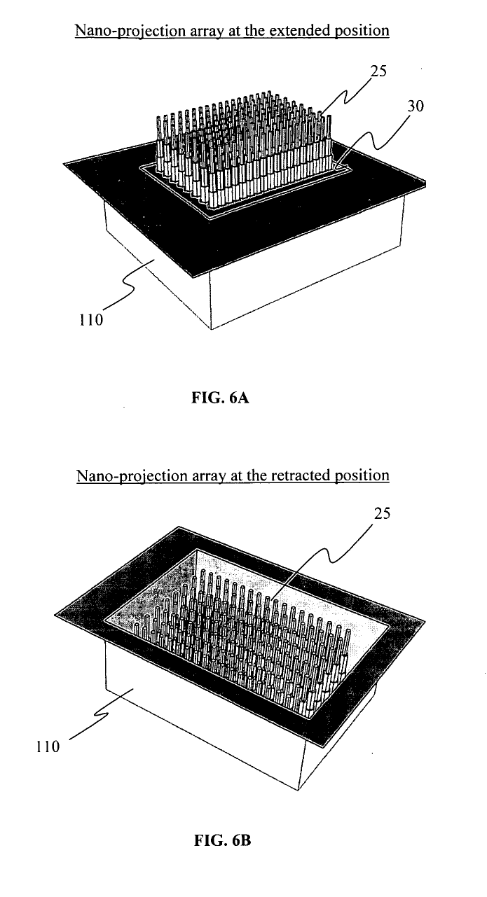

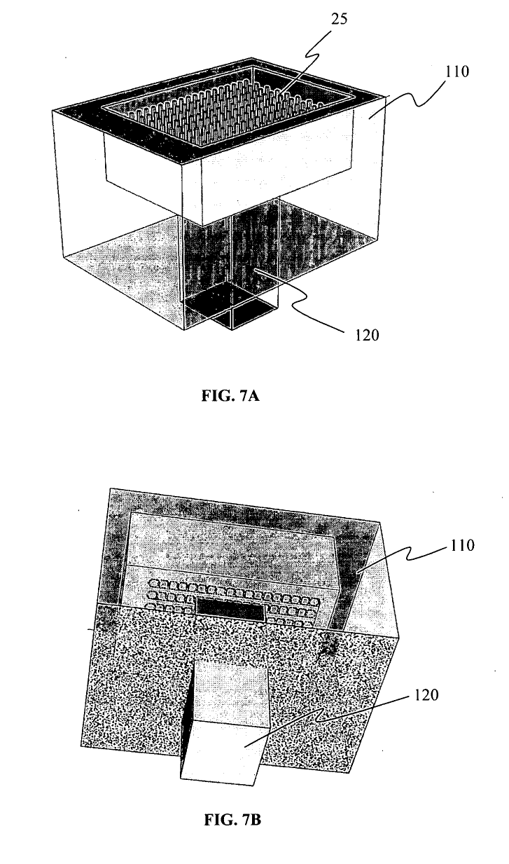

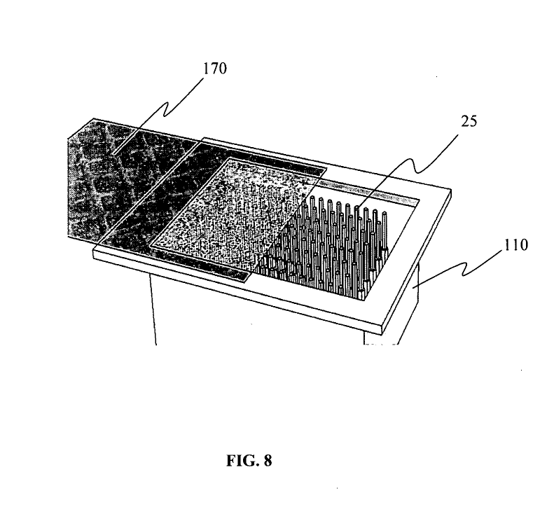

FIG. 6A shows the nano-projection array 20 at the extended position and FIG. 6B shows the nano-projection array 20 at the retracted position. In addition, FIG. 7A, FIG. 7B, and FIG. 8 show the nano-projection array 20 disposed within the peripheral structure 110 at the retracted position.

In many embodiments, the nano-projections 25 of the nano-projection array 20 are disposed (a) within, or substantially within, the peripheral structure 110 when the nano-projection array 20 is at the retracted position; and (b) external, or substantially external, the peripheral structure 110 when the nano-projection array 20 is at the extended position. As shown in FIG. 6A, the first ends 35 of the nano-projections 25 are external the peripheral structure 110 (i.e., located above the plane of the top surface of the peripheral structure 110) when the nano-projection array 20 is at the extended position. As shown in FIG. 6B, the first ends 35 of the nano-projections 25 are positioned at, or below, the top surface of the peripheral structure 110 when at the retracted position.

The displacement of the nano-projection array 20 from the retracted position to or toward the extended position facilitates or effectuates insertion of the nano-projections 25 through the SC and into the epidermis or other bodily tissue.

In various embodiments, the displaceable carrier 120 is shaped, dimensioned, and/or configured such that a distance of displacement thereof results in a corresponding distance of displacement of the nano-projection array 20 that is coupled thereto, carried thereby, or associated therewith. Accordingly, in various embodiments, the distance of displacement of the displaceable carrier 120 corresponds, or substantially corresponds, to a distance of displacement of the nano-projections 25 and a distance at which the nano-projections 25 are inserted into the body.

In several embodiments of the present disclosure, the displaceable carrier 120 can be biased to be disposed at, or towards, the first or retracted position. In such embodiments, the displaceable carrier 120 can be configured to return to first position or retracted position after a displacement to the second or extended position.

In some embodiments, the displaceable carrier 120 includes, or is, a resilient or biased deformable substrate or platform. In particular embodiments, the displaceable carrier 120 includes, or is coupled to, at least one resilient or biasing element that facilitates or effectuates the biasing of the displaceable carrier 120 at the first or retracted position. In other words, the displaceable carrier 120 can include, or be coupled to, at least one resilient or biasing element that facilitates or effectuates automatic, or substantially automatic, return of the displaceable carrier 120 to the first or retracted position after a displacement thereof to or toward the second or extended position.

### Displacement Control Element(s)

In most embodiments of the present disclosure, one or more compositions (e.g., vaccines) can be delivered to a predictable or controlled depth within the skin, for instance to the epidermis or dermis. In other words, a distance of displacement or insertion of the nano-projections 25 into the body can be controlled or managed.

In many embodiments, the control of the insertion of nano-projections 25 into the body is facilitated or effectuated by a control of the displacement of the displaceable carrier 120 that carries, or is coupled to, the nano-projections 25. This is to say, the displacement of the displaceable carrier 120 can be controlled to therefore control the insertion of the nano-projections 25 into the body. For example, in particular embodiments, the distance of displacement of the displaceable carrier 120 determines a distance of displacement of the nano-projection array 20, and hence a distance of displacement of the nano-projections 25 carried by, coupled to, or associated with the displaceable carrier 120.

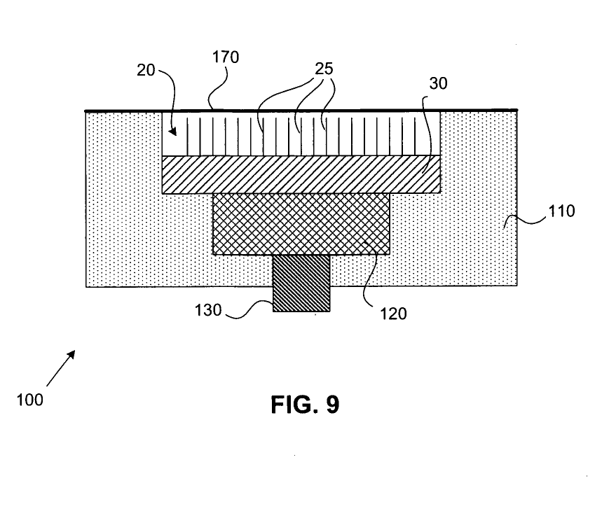

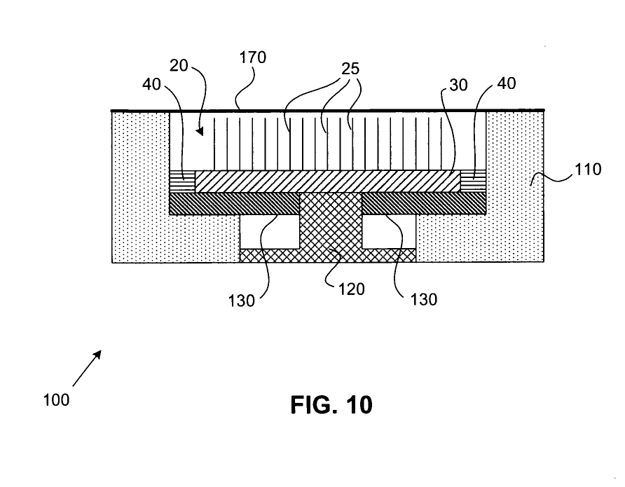

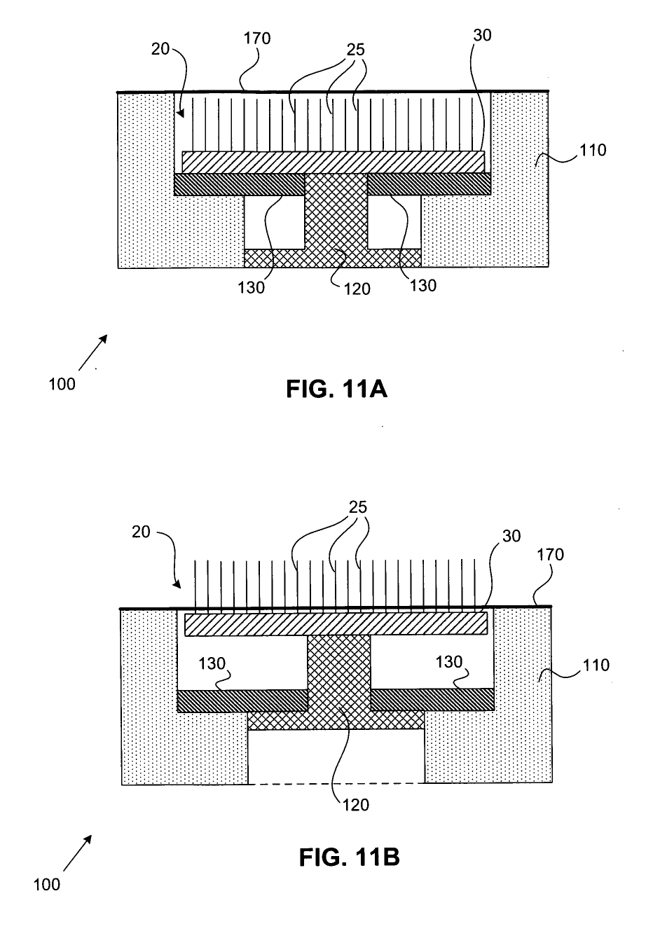

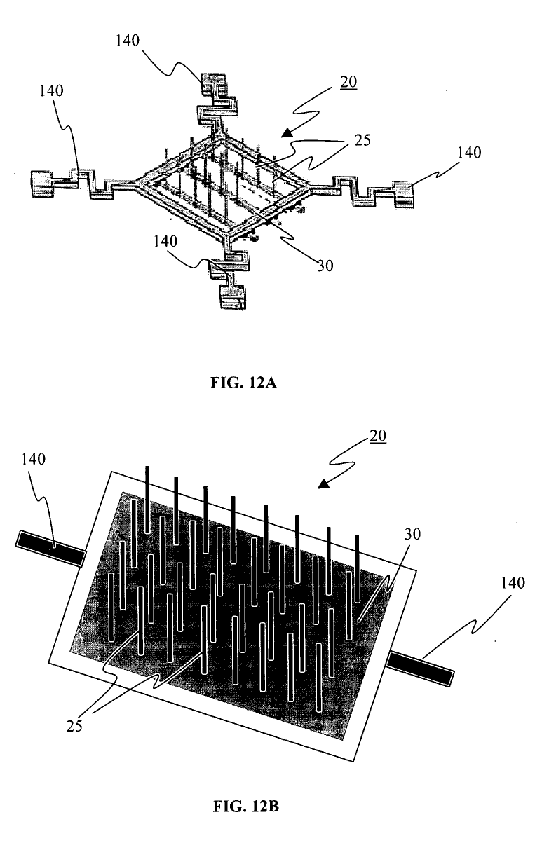

FIG. 9, FIG. 10, FIG. 11A and FIG. 11B show particular application units 100 that include a displacement control element or structure 130 (or displacement limiting element) for controlling the displacement of the displaceable carrier 120 and hence displacement of the nano-projection array 20.

In several embodiments of the present disclosure, at least a portion of the displacement control element 130 is coupled to, carried by, or housed within a portion of the displaceable carrier 120. As shown in FIG. 9, in some embodiments, the displacement control element 130 can be coupled to the displaceable carrier 120 (e.g., coupled to a base surface of the displaceable carrier 120).

The displacement control element 130 is shaped and/or configured to control or limit the displacement of the displaceable carrier 120. More specifically, in several embodiments, the displacement control element 130 is configured to control a distance of displacement of the displaceable carrier 120.

In some embodiments of the present disclosure, for instance as shown in FIG. 10, FIG. 11A and FIG. 11B, the displacement control element 130 is coupled to, carried by, or disposed within the peripheral structure 110 or housing. The displacement control element 130 can be a mechanical structure that is shaped and/or configured to control or limit the displacement of the displaceable carrier 120. For instance, the displacement control element 130 can be a rigid, or substantially rigid, unit or structure(s) that controls (e.g., limits or prevents) the displacement of the displaceable carrier 120.

In particular embodiments, the placement of the displacement control element 130, more specifically the rigid unit, prevents further displacement of the displaceable carrier 120 when the displaceable carrier 120 makes contact with the displacement control element 130. In specific embodiments, the displacement control element 130, more specifically the rigid unit(s), are positioned and configured to define, substantially define, demarcate, or substantially demarcate, the first position (or retracted position) and/or the second position (or extended position) of the displaceable carrier 120.

FIG. 11A shows a displacement or position of the displaceable carrier 120 at the first (or retracted) position and FIG. 11B shows a displacement or position of the displaceable carrier 120 at the second (or extended) position. As shown in FIG. 11A and FIG. 11B, the displacement control element 130 facilitates a prevention of further displacement of the displaceable carrier 120 past the second (or extended) position in a direction away from the first position.

In various embodiments, the displacement control element 130 includes a set of tensioned elements (not shown), for example mechanical springs, deformable membranes, and/or displaceable levers, that is configured to facilitate or effectuate the control of the distance of displacement of the displaceable carrier 120. The set of tensioned elements can be coupled to, carried by, or partially housed within the displaceable carrier 120. For example, the set of tensioned elements can be coupled to a side, an edge, or a corner of the displaceable carrier 120.

### Force Management Elements

FIG. 10, FIG. 12A and FIG. 12B show aspects of particular embodiments including a set of force management elements 140 that is coupled to a nano-projection array 20.

In particular embodiments, for instance as shown in FIG. 12A, the set of force management elements 140 can be coupled to the corners or periphery of the nano-projection array 20. It will however be understood that the set of force management elements 140 can be alternatively disposed, position, or coupled to the nano-projection array 20. For instance, in specific embodiments, the set of force management elements 140 can be soldered to the nano-projection array 20 at one or more locations.

The set of force management elements 140 is disposed and/or configured to control a force or pressure that is applied, or transferred, to the nano-projection array 20. In several embodiments, the set of force management elements 140 is disposed and/or configured to control a force or pressure that is transferred from the displaceable carrier 120 to the nano-projection array 20.

In various embodiments, the set of force management elements 140 is disposed and/or configured to control, distribute, or limit a force or pressure applied, or transferred, to the nano-projection array 20. For instance, in particular embodiments, the set of force management elements 140 is configured to uniformly, or substantially uniformly, distribute a force that is applied, or transferred, to the nano-projection array 20, and hence to the nano-projections 25 of said nano-projection array 20. In several embodiments, the control, distribution, or limitation of a force that is applied, or transferred, to the nano-projections 25 facilitates or effectuates an insertion of the nano-projections 25 into the body at a uniform, or substantially uniform, pressure and/or depth.

The set of force management elements 140 can include a number of tensioned elements, for instance at least two, four, six, ten, or more tensioned elements. The tensioned elements can include, for example, springs (e.g., micro-mechanical springs). Alternatively, the tensioned elements can include displaceable levers or displaceable support arms.

In various embodiments, for instance as shown in FIG. 12A, the set of force management elements 140 includes four tensioned elements (e.g., springs) that are disposed at each corner of a square-shaped nano-projection array 20. In other embodiments, for instance as shown in FIG. 12B, the set of force management elements 140 includes two tensioned elements that are disposed at opposite sides of the nano-projection array 20. In certain embodiments, the tensioned elements can be coupled to a frame or support that itself is coupled to the nano-projection array 20.

### Fluidic Channel/Fluidic Reservoir

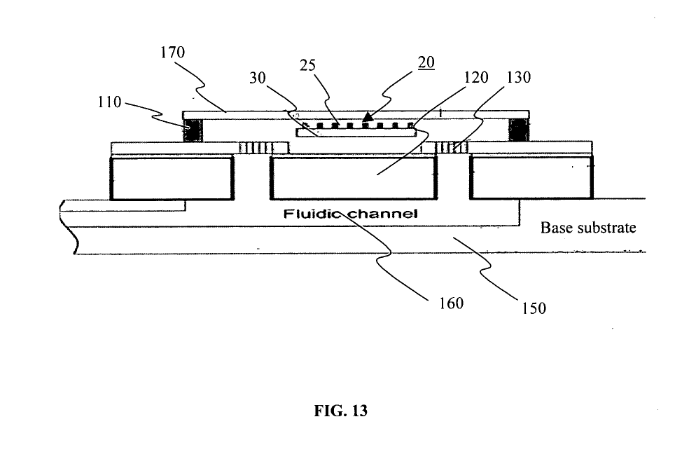

FIG. 13 shows an application unit 100 that further includes a base substrate 150 (also known as a polymer-based substrate) with a fluidic channel 160 or fluidic reservoir 160 formed, or embedded, therein according to various embodiments of the present disclosure.

In the present disclosure, application units 100 that include a fluidic reservoir 160 are used in association with nano-projection arrays 20 wherein the nano-projections 25 are, or include, nano-needles or nano-tubes. Further description of nano-projection arrays 20 wherein the nano-projections 25 include nano-needles or nano-tubes will be provided below.

The fluidic reservoir 160 is shaped and configured to hold or store a predetermined volume of the composition (e.g., vaccine). The volume of the composition that is held or stored within the fluidic reservoir 160 can depend upon particular structural or functional characteristics of the apparatus and/or a composition dose objective under consideration.

In several embodiments, the fluid reservoir 160 has a depth (or height) of between approximately 100 μm and 250 μm. In various embodiments, the fluid reservoir 160 has a depth (or height) of between approximately 125 μm and 200 μm, for instance approximately 150 μm, 160 μm, or 175 μm. In some embodiments, a cross-sectional area of the fluid reservoir 160 is between approximately 1 mm² and 5 cm². In various embodiments, a cross-sectional area of the fluid reservoir 160 is between approximately 5 mm² and 2.5 cm², for instance approximately 7.5 mm², 1 cm², or 2 cm².

In many embodiments, the base substrate 150 is disposed adjacent the peripheral structure 110 of the application unit 100. More specifically, in numerous embodiments, the base substrate 150 is disposed at a bottom side of the peripheral structure 110. In several embodiments, the fluidic reservoir 160 is disposed adjacent to the displaceable carrier 120 and/or the nano-projection array 20.

In many embodiments, the composition held or stored within the fluidic reservoir 160 can be delivered, for instance communicated, by or through the nano-projections 25 (i.e., nano-needles) to the target site during insertion of the nano-projections 25 to the target site. Further details with regard to the delivery or administration of a composition held or stored within the fluidic reservoir 160 using nano-needles will be provided below.

### Sealing Film

In many embodiments, a sealing film 170 (also known as a sealing polymer film or polymer film) can be coupled, adhered or attached to the peripheral structure 110. More specifically, the sealing film 170 can be attached, assembled, or adhered to the top (i.e., the top side) of the peripheral structure 110. Adhesives, or adhesive materials, for example pressure sensitive (PSA) or removable adhesives, can be used to attach, assemble, or adhere the sealing film 170 to the peripheral structure 110.

Attachment, assembly, or adhesion of the sealing film 170 to the top of the peripheral structure 110 can facilitate or effectuate an isolation of the nano-projection array 20 that is disposed within the peripheral structure 110. Accordingly, the attachment, assembly, or adhesion of the sealing film 170 to the top of the peripheral structure 110 can help to maintain the nano-projections 25 disposed within the peripheral structure 110 in a sterile state prior to their use (e.g., insertion into the body).

In some embodiments, the sealing film 170 remains adhered to the peripheral structure 110 during displacement and insertion of the nano-projections 25 into the body. Accordingly, the nano-projections 25 pass, or pierce, through the sealing film 170 when inserted into the body. In other embodiments, the sealing film 170 is removed from the peripheral structure 110 prior to displacement and insertion of the nano-projections 25 into the body.

### Modes of Administering or Delivering a Composition

As above-mentioned, the displacement and insertion of the nano-projections 25 into a target site or tissue within the body, for instance to the epidermis, facilitates or effectuates delivery of the composition (e.g., vaccine) to the target site. The nano-projections 25 are shaped, dimensioned, and/or configured to aid or enable the delivery of the composition (e.g., vaccine) to the target site. Embodiments of the present disclosure facilitate or effectuate control of the insertion of the nano-projections 25 into the body. More specifically, particular embodiments facilitate or effectuate preferential insertion of the nano-projections 25 to specific body tissue or skin layer (e.g., the epidermis) while avoiding, or generally avoiding, other body tissues or skin layers (e.g., the dermis).

### Administering or Delivering a Composition Using Solid Nano Projections (Nano-Rods)

In some embodiments of the present disclosure, at least a portion of the nano-projections 25 of each nano-projection array 20 are solid (i.e., non-hollow). Solid nano-projections 25 can be hereinafter referred to as nano-rods 25a or nano-wires 25a. In addition, a nano-projection array 20 that includes nano-rods 25a can be referred to as a nano-rod array 20a.

A composition (e.g., vaccine) can be coated onto at least a portion of the surface of the nano-rods 25a for delivery to the target site in association with (e.g., during) insertion of the nano-rods 25a to the target site. Nano-rods 25a with a volume of the composition coated on at least a portion of their surface can be referred to as coated nano-rods 25a.

In numerous embodiments, a surface area of the nano-rods 25a coated with the composition (e.g., vaccine) can be selected and/or varied, for instance depending upon a type of composition, length of the nano-rods 25a, and/or dosage of the composition that is required.

In particular embodiments, the composition coated onto the nano-rods 25a is a lyophilized form of a vaccine, which is originally prepared as suspensions of one or more antigens together with excipients, adjuvants, and/or stabilizers (e.g., alum, mannitol, chitosan and dextran). The vaccine includes antigens that are capable of eliciting an immune response against a human pathogen. For example, the vaccine can be a vaccine against the human herpes virus, hepatitis B virus, hepatitis A virus, or influenza virus.

As mentioned above, in various embodiments, the nano-projections 25, for instance the nano-rods 25a, are configured to be of a length capable of reaching the target site for delivery of the composition. In numerous embodiments, the insertion or injection of the coated nano-rods 25a into the body, for instance to the epidermis of the skin, brings the composition into physical contact with, or proximity of, the target site, for instance epidermal cells. The transport of the composition to such cells by way of nano-projection displacement, and the resulting physical contact of the composition with the target site (e.g., epidermis), facilitates or effectuates delivery of the composition to the target site.

In certain embodiments, the insertion or injection of the coated nano-rods 25a into the body brings the composition into physical contact with, or proximity of, dendritic cells in the dermis. The transport of the composition into physical contact with, or proximity of, the dendritic cells in the dermis facilitates or effectuates delivery of the composition to thereto.

In many embodiments, the ability to use coated nano-rods 25a for delivering vaccines removes the necessity of a cold chain, i.e., maintaining vaccines to be delivered at specific temperature ranges, for instance at a temperature of between approximately 2° C. and 8° C. More specifically, the ability to coat nano-rods 25a with lyophilized forms of vaccines removes the necessity for a cold chain, which can be particularly relevant, or useful, in developing countries wherein it may be difficult to store vaccines, and apparatuses for delivery of such vaccines, at specific temperature ranges (e.g., low temperature ranges).

### Administering a Composition Using Hollow Nano Projections (e.g., Nano-Needles or Nano-Tubes)

In some embodiments of the present disclosure, at least a portion of the nano-projections 25 of each nano-projection array 20 are hollow. In other words, in some embodiments of the present disclosure, at least a portion of the nano-projections 25 of each nano-projection array 20 includes a channel 70 (as shown in FIG. 1D) formed therethrough. A hollow nano-projection 25, or a nano-projection 25 that includes the channel 70 formed therethrough, can be referred to as a nano-needle 25b or a nano-tube 25b. In addition, nano-projection arrays 20 that include nano-needles 25b can be referred to as nano-needle arrays 20b.

The channel 70 of a nano-needle 25b is sized and configured to allow communication of a composition (e.g., a vaccine) into and through the nano-needle 25b (e.g., from the second end 40 to the first end 35 of the nano-needle 25b). In various embodiments, the diameter of the channel 70 of a nano-needle 25b can be selected and varied, for instance depending upon type of composition delivered by the apparatus, size of molecules of the composition delivered by the apparatus, and/or size or configuration of the nano-needle 25b. In specific embodiments, the channel 70 can be configured to facilitate or effectuate control of communication of the composition through the channel 70, for example control of volume of the composition communicated through the channel 70.

In several embodiments, the administration or delivery of the composition to the target site using a nano-needle 25b occurs in association with the insertion of the nano-needle 25b to the target site within the body (e.g., the epidermis or dermis). More specifically, communication of the composition through the channel 70 of the nano-needle 25b occurs during, or subsequent, to the displacement of the nano-needle 25b to the target site, thereby enabling delivery of the composition at the target site.

As mentioned above, nano-projection arrays 20 including nano-needles 25b (i.e., nano-needle arrays 20b) are used in association, or together, with application units 100 that include fluidic reservoirs 160. In many embodiments of the present disclosure, the channels 70 of nano-needles 25b are in fluid communication with the application unit's fluidic reservoir 160. In numerous embodiments, the fluid reservoir 160 is formed or configured in such a way that no air bubbles are present between the fluid reservoir 160 and the channels 70 of the nano-needles 25b.

The composition that is held or stored within the fluidic reservoir 160 can be communicated or transported through the channels 70 of the nano-needles 25b (e.g., from the second end 40 to the first end 35 of the nano-needles 25b) for delivery to the target site within the body when the nano-needles 25b are inserted to the target site. More specifically, the composition is communicated from fluidic reservoir 160 from the second end 40 of the nano-needles 25b to the first end 35 of the nano-needles 25b, and released at the second end 40 to the target site.

The displacement of the displaceable carrier 120 from the retracted position to the extended position causes a corresponding displacement of the nano-needle array 20b, and hence nano-needles 25b of said nano-needle array 20b, from the retracted position to the extended position. Displacement of the nano-projection array 20, and hence nano-needles 25b, to the extended position facilitates or effectuates insertion of the nano-needles 25b to the target site.

In addition, in various embodiments, the displacement of the displaceable carrier 120 from the retracted position to the extended position triggers, facilitates, or effectuates a simultaneous, or substantially simultaneous, communication of the composition stored within the fluidic reservoir 160 through the channels 70 of the nano-needles 25b for delivery to the target site.

In particular embodiments, the communication of the composition from the fluidic reservoir 160 through the channels 70 of the nano-needles 25b for release at the target site can be controlled. For instance, in selected embodiments, a volume of composition communicated from the fluidic reservoir 160 to the target site can be selected and/or varied.

### Particular Dosages of Compositions

In embodiments of the present disclosure wherein the nano-projections 25 are nano-needles 25b, the insertion of the nano-needles 25b to the target site and communication of the composition through the channels 70 of said nano-needles 25b to the target site can facilitate or effectuate the delivery of a dose, for instance an effective dose, of the composition to the target site. Similarly, in embodiments of the present disclosure wherein the nano-projections 25 are nano-rods 25a, the insertion of the coated nano-rods 25a to the target site brings the composition into physical contact with the target site to thereby facilitate or effectuate delivery of a dose, for instance an effective dose, of the composition to the target site.

For purposes of the present disclosure, a dose or a dosage refers to a specified quantity (i.e., amount or volume) of a biological, pharmaceutical, or chemical composition, for example a vaccine, that is administered or delivery in a single or sequential (in case of multiple nano-projection arrays) application (e.g., transdermal delivery). In addition, an effective dose or dosage refers to a minimum quantity (i.e., amount or volume) of a biological, pharmaceutical, or chemical composition, for example vaccine, that is capable of inducing, providing, or producing an effective therapeutic, protective, immunomodulatory, or immunogenic response within the body.

In various embodiments of the present disclosure, an effective therapeutic, protective, immunomodulatory, or immunogenic response can be produced, provided, or induced with a delivery of a lower dosage (or dose) of the composition than would be applicable to prior composition delivery techniques.

As above described, in various embodiments wherein the composition is a vaccine, the administration or delivery of the vaccine to the epidermis presents the antigenic, immunogenic, or like bioactive agent (hereinafter referred to as an active agent) of the vaccine to the immune cells that reside in the epidermis (more specifically the Langerhans cells) or dermis (more specifically dendritic cells).

In numerous embodiments, the presentation of the active agent of the pharmaceutical or chemical composition (e.g., vaccine) to the Langerhans cells within the epidermis results in inducement, production, or generation of a therapeutic or immunogenic response by the body.

In several embodiments of the present disclosure, an effective therapeutic, protective, or immunogenic response can be produced, provided, or induced with a single or multiple (boostering) dose of the composition (e.g., vaccine). Delivery of a composition in accordance with embodiments of the present disclosure can provide an effective therapeutic, protective, or immunogenic response at a lower dose of the composition as compared to doses that are associated with conventional methods for delivering pharmaceutical or chemical compositions (e.g., via intra-muscular injections or oral delivery). In addition, an effective therapeutic, protective, or immunogenic response can be produced, provided, or induced with or without a need for booster immunizations (or booster doses).

The effective dosage in accordance with numerous embodiments of the present disclosure can be at least approximately 10% lower than the dosages used with conventional administration or delivery methods, for example intramuscular or intravenous pharmaceutical delivery methods. The effective dosage in accordance with various embodiments of the present disclosure can be at least approximately 25% lower than dosages required with conventional pharmaceutical administration or delivery methods. More specifically, the effective dosage in accordance with particular embodiments is at least approximately 50% lower, for example approximately 60%, 65%, 70%, 75%, or more, as compared to dosages required with conventional administration or delivery methods.

The effective dosage of the composition according to several embodiments of the embodiment can be administered or delivered with a single injection or insertion of the nano-projections 25 of one nano-projection array 20 (or one set of nano-projections 25) into the body, for instance to the target site within the body. Alternatively, the effective dosage of the composition can be administered or delivered with an injection or insertion (e.g., a simultaneous injection or insertion) of at least two sets of nano-projections 25 into the body. In particular embodiments, at least two different compositions can be simultaneously delivered into the body using at least two corresponding sets of nano-projections 25.

### Systems Including Multiple Nano-Projection Arrays 20 (Sets of Nano-Projections)

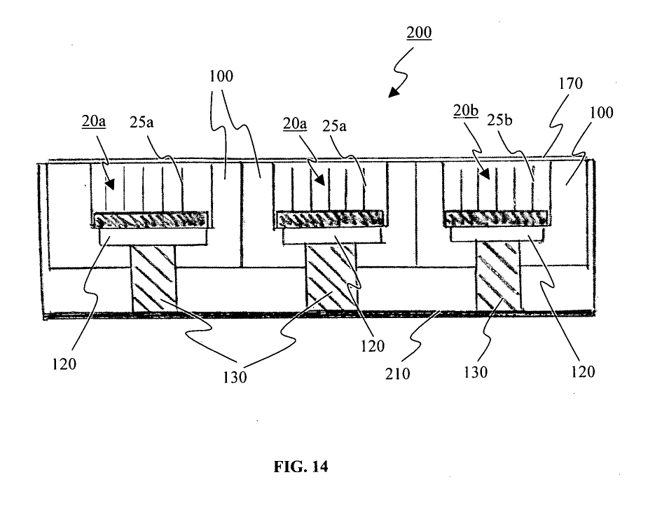

FIG. 14 is a schematic representation of a system 200 that includes at least two nano-projection arrays 20 as according to particular embodiments of the present disclosure.

Systems 200 that include at least two, for example two, four, ten, or more, nano-projection arrays 20 are provided in accordance with particular embodiments of the present disclosure.

In several embodiments, each nano-projection array 20 of a particular system 200 is identical, or substantially similar, to each other. In other embodiments, for example as shown in FIG. 14, the system 200 includes different types of nano-projection arrays 20, for instance at least one nano-rod array 20a with nano-rods 25a and at least one nano-needle array 20b with nano-needles 25b.

In addition, the system 200 of certain embodiments can also include one or more conventional needle array(s) or chip(s), for example a micro-needle array together with one or more nano-needle arrays 20 of the present disclosure.

The at least two nano-projection arrays 20 of the system 200 can be configured or disposed in an ordered array, for instance in ordered rows. In most embodiments, the system 200 also includes a number of application units 100. In many embodiments, each nano-projection array 20 is coupled to, carried by, or associated with one application unit 100. For instance, in several embodiments, each nano-projection array 20 is coupled to, carried by, or associated with at least a part of one application unit 100.

In several embodiments, the system 200 includes a connecting substrate or structure 210 (also known as a linking substrate or structure) that is configured to connect, or couple, the multiple nano-projection arrays 20 and the number of application units 100 to each other. In a number of embodiments, the connecting structure 210 is configured and disposed to inter-couple or connect the peripheral structures 110 and/or the displaceable carriers 120 of the application units 100 of the system 200.

In some embodiments, the multiple nano-projection arrays 20 of a particular system 200 can simultaneously administer or deliver a composition to one or more target sites; tissues, or skin layers within the body. For example, in certain embodiments, a particular system 200 can include a first nano-projection array 20 including nano-projections 25 of a length suitable for reaching a first target site (e.g., the epidermis) as well as a second nano-projection array 20 including nano-projections 25 of a length suitable for reaching a second, different, target site (e.g., the dermis). In particular embodiments, the nano-projections 25 of each of the multiple nano-projection arrays 20 of the system 200 can be simultaneously inserted into the body for administering the composition to one or more target sites, body tissue, or skin layers within the body.

In some embodiments, the connecting structure 210 is configured and/or disposed such that an application of force or pressure onto the connecting structure 210 results in a simultaneous, or substantially simultaneous, application of force or pressure onto the displaceable carriers 120 of the number of application units 100 of the system 200. In other words, the force applied onto the connecting structure 210 can be distributed for simultaneous transfer to each displaceable carrier 120 of the number of application units 100 that are coupled to, or carried by, the connecting structure 210.

The simultaneous application, or transfer, of force or pressure to each of the displaceable carriers 120 triggers, facilitates, or effectuates simultaneous, or substantially simultaneous, displacement of displaceable carriers 120 of the system 200 to thereby facilitate or effectuate simultaneous displacement of each of the nano-projection arrays 20 of the system 200. The simultaneous displacement of each nano-projection array 20 thereby aids or enables a simultaneous insertion of the nano-projections 25 of each nano-projection array 20 into the body, for instance to the epidermis.

Although the embodiments described above pertain to a simultaneous displacement of multiple nano-projection arrays 20, it will be understood that an ability to control the application of force or pressure for displacing the displaceable carriers 120 of a particular system 200 in a consecutive or sequential manner to thereby displace the nano-projection arrays 20 in a correspondingly consecutive or sequential manner is also included within the scope of the present disclosure.

In some embodiments of the present disclosure, the compositions (e.g., vaccines) that are associated with, or delivered by, two or more nano-projection arrays 20 of a particular system 200 are identical, or substantially similar.

In other embodiments, the compositions (e.g., vaccines) that are associated with, or delivered by two or more nano-projection arrays 20 of a particular system 200 are different. In other words, in some embodiments, a first nano-projection array 20 of a system 200 can be used to deliver a first composition (or a first type of vaccine) and a second nano-projection array 20 of said system 200 is used to deliver a second composition (or a second type of vaccine), each of the first and second compositions (or the first and second types of vaccines) being different from each other.

In particular embodiments, the number and/or type of compositions (e.g., vaccines) that can be delivered using a particular system 200 can be selected and/or varied, for instance depending upon particular structural or functional characteristics of the system 200 and/or a clinical situation under consideration.

### Systems Including Multiple Nano-Needle Arrays 20b for Delivering Multiple Compositions

As described above, in several embodiments of the present disclosure, the nano-projections 25 of nano-projection arrays 20 can include nano-needles 25b (said nano-projection arrays 20 known as nano-needle arrays 20b).

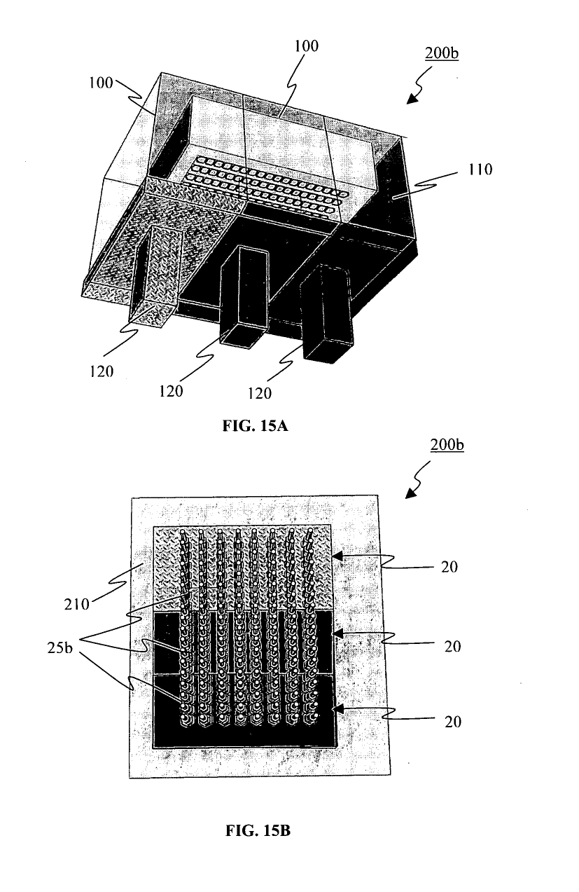

FIG. 15A and FIG. 15B show a system 200b that includes multiple nano-needle arrays 20b as according to particular embodiments of the present disclosure.

In many embodiments, the system 200b that includes multiple nano-needle arrays 20b also includes a corresponding number of application units 100, which include fluidic reservoirs 160. The fluidic reservoirs 160 store compositions, which can be similar or different from each other, that can be communicated through the channels 70 of the nano-needles 25b of respective nano-needle arrays 25b for delivery to the target site.

In certain embodiments, for instance as shown in FIG. 14A and FIG. 14B, the system 200b can include three nano-needle arrays 20b and three application units 100, each with a separate fluidic reservoir 160 that is fluidly communicable with the channels 70 of the nano-needles 25b of a corresponding nano-needle array 20b.

In the system 200b as shown in FIG. 14A and FIG. 14B, each fluidic reservoir 160 of the three application units 100 is fluidly isolated and holds or stores a different composition (e.g., vaccine) from each other.

It will be understood that in certain alternative embodiments, the fluidic reservoirs 160 of different application units 100 of a system 200b can be fluidly linked or interconnected, for instance via a linkage passage (or a fluid linkage passage) (not shown). Interconnected fluidic reservoirs 160 of a particular system 200b can store identical, or substantially similar, compositions (e.g., vaccines).

In specific embodiments, the system 200b includes one fluidic reservoir 160 that is fluidly communicable with the channels 70 of the nano-needles 25 of each nano-needle array 20b of the system 200b. In addition, in particular embodiments, the system 200b can have one peripheral structure 110 that is shaped, dimensioned, and/or configured to surround the one fluidic reservoir 160, and each nano-needle array 20b of the system 200b.

### Methods, Processes, and Techniques of Manufacture

Methods, processes, and techniques for manufacturing particular apparatuses and systems are also provided by various embodiments of the present disclosure.

### Processes for Manufacturing an Apparatus

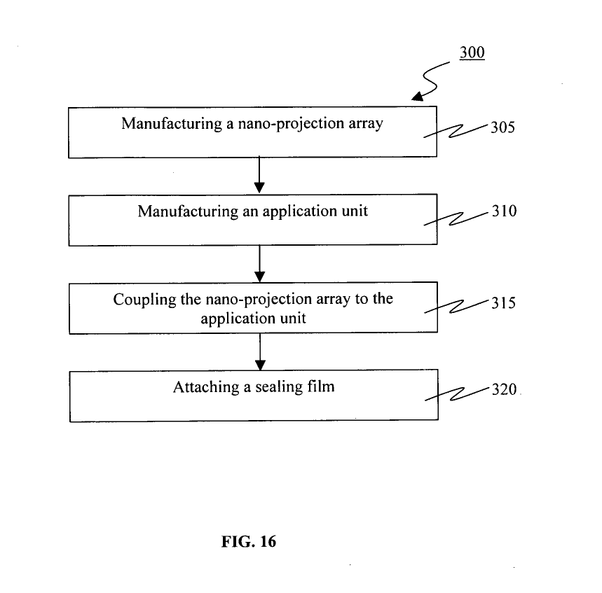

FIG. 16 shows a flowchart of a process 300 for manufacturing an apparatus as according to particular embodiments of the present disclosure.

In a first process portion 305, a nano-projection array 20 is fabricated, synthesized, or manufactured. As above-mentioned, each nano-projection array 20 includes a number of nano-projections 25 that are carried or supported by a carrier substrate 30 or carrier medium 30.

In many embodiments of the present disclosure, the fabrication of a nano-projection array 20 includes growth, synthesis, or construction of a set of nano-projections 25 on a carrier substrate 30 or carrier medium 30.

The growth of cylindrical nano-projections 25 on a carrier substrate 30 can be performed using a procedure or technique described in C. Li, G. Fang, Q. Fu, F. Su, G. Li, X. Wu, X. Zhao, Effect of substrate temperature on the growth and photoluminescence properties of vertically aligned ZnO nanostructures, Journal of Crystal Growth, 2006, 292, page 19-25.

While the procedure described in C. Li et al. results in production of cylindrical nano-projections, in many embodiments of the present disclosure, conical, tapered, layered, or segmented nano-projections 25 are instead formed.

In several embodiments, vertically aligned nano-projections 25 can be formed or synthesized using a vapour-solid (VS) mechanism on a silicon carrier substrate 30 that is coated with a Zinc oxide seed later of approximately 200 nm. C. Li et al. discloses the formation or synthesis of nano-rods via a single growth step, thereby forming nano-rods of a single segment. However, in various embodiments of the present disclosure, the formation or synthesis of the nano-projections 25 occurs via a number of repeated growth or synthesis steps, for example at least two, three, four, five, or more growth steps. In addition, in certain embodiments, a homoepitaxial anisotropic growth process is utilized for synthesizing the nano-projections 25.

Further details of formation, synthesis, or manufacture of the nano-projection arrays 20 are provided in the examples (i.e., examples one and two) provided below. More specifically, further description of the manufacture of nano-projection arrays 20 that include nano-rods 25a (i.e., manufacture of nano-rod arrays 20a) is provided in example one below, and further description of the manufacture of nano-projection arrays 20 that include nano-needles 25b (i.e., manufacture of nano-needle arrays 20b) is provided in example two below.

A second process portion 310 involves assembling, manufacturing, or fabricating, an application unit 100. As above described, in many embodiments, an application unit 100 includes a peripheral structure 110 and a displaceable carrier 120. In several embodiments, the application unit 100 further includes a displacement control element 130 and a set of force management elements 140.

In numerous embodiments, the displaceable carrier 120 is coupled to the peripheral structure 110, and is configured for displacement relative to the peripheral structure 110. In several embodiments, the displaceable carrier 120 is shaped and configured to be disposed within, or at least partially within the peripheral structure 110. In various embodiments, the displaceable carrier 120 is configured to transfer a force that is applied thereto to a nano-projection array 20.

In many embodiments, the displacement control element 130 is disposed and/or configured in a manner for facilitating or effectuating control of the displacement of the displaceable carrier 120 relative to the peripheral structure 110. In several embodiments, the displacement control element 130 is coupled to, carried by, or housed within the displaceable carrier 120. In various embodiments, the displacement control element 130 is coupled to, carried by, or housed within the peripheral structure 110 to control, for instance physically limit or prevent, the displacement of the displaceable carrier 120 relative to the peripheral structure 110. In some embodiments, the displacement control element 130 is configured to allow and/or control transfer of a force to the displaceable carrier. In various embodiments, the displacement control element 130 is configured to control the force transferred from the displaceable carrier 120 to the nano-projection array 20.

In a third process portion 315, the nano-projection array 20 is coupled to the application unit 100. In many embodiments, the nano-projection array 20 is disposed within, or substantially within, the peripheral structure 110 or peripheral housing 110 of the application unit 100. In many embodiments, the nano-projection array 20 is carried by the displaceable carrier 120 of the application unit 100.

In particular embodiments, specific portions, or elements, of a nano-projection array 20 can be soldered, adhered, welded, or molded to the application unit 100. For instance, in specific embodiments, the nano-projection array 20 can be soldered or adhered to a set of force management elements 140. Other methods, techniques, or means can be used to couple a nano-projection array 20 to an application unit 100 in accordance with the scope of the present disclosure.

In a fourth process portion 320, the sealing film 170 (e.g., a polymer sealing film), is coupled, applied, or assembled, to the application unit 100 to seal off, or isolate, the nano-projection array 20 that is carried by the application unit 100. In several embodiments, the sealing film 170 is adhered to a top of the peripheral structure 110 to isolate the nano-projection array 20 that is disposed within the peripheral structure 110. Adhesives, or adhesive materials, for example pressure sensitive (PSA) or removable adhesives, can be used to adhere or stick the sealing film 170 onto the top of the peripheral structure 110.

In several embodiments, the adhesion of the sealing 170 onto the top of the peripheral structure 110 to isolate the nano-projection array 20 that is disposed within the peripheral structure 110 helps to maintain sterility of the nano-projection array 20 until the moment of usage (e.g., displacement and insertion of the nano-projections 25 into the body).

### Processes for Manufacturing a System

As above described, systems 200 of particular embodiments of the present disclosure include multiple nano-projection arrays 20 with a number of application units 100.

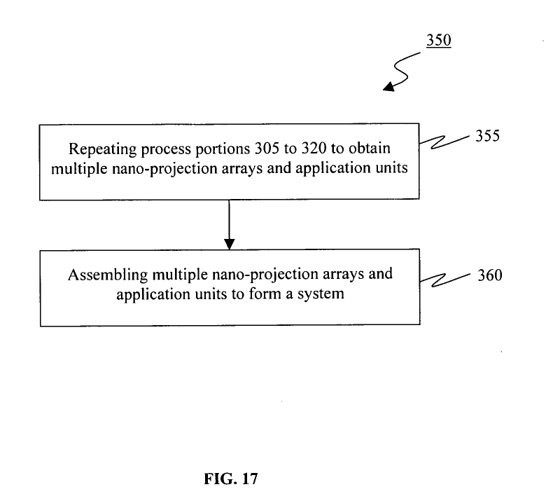

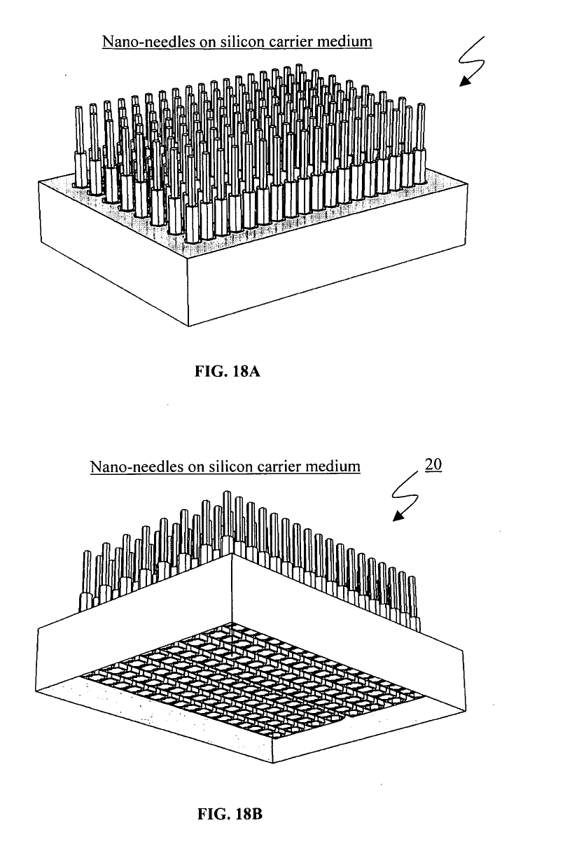

FIG. 17 shows a flowchart of a process 350 for manufacturing a system 200 that includes multiple nano-projection arrays 20 with a number of application units 100 according to an embodiment of the present disclosure.

In many embodiments, the process 350 includes each of the above mentioned process portions 305 to 320. More specifically, a first process portion 355 of the process 350 involves repeating the process portions 305 to 320a number of times (e.g., three or more times) to manufacture a system 200 that includes said number (e.g., three or more) of nano-projection arrays 20 and application units 100.

A second process portion 360 involves assembling, coupling, or connecting the number (e.g., three or more) of nano-projection arrays 20 and application units 100 to each other. In several embodiments, the connecting structure 210 is used for assembling, coupling, or connecting the nano-projection arrays 20 and application units 100 to each other.

### Material(s) Used for Manufacturing Apparatuses and Systems

Apparatuses and systems of particular embodiments of the present disclosure can be constructed using a variety of materials. In many embodiments, the apparatus and systems are constructed using biocompatible materials such as titanium, gold, silver or silicon. In some embodiments, the entire apparatus or system (i.e., each component of the apparatus or system) is made from biocompatible materials. Alternatively, in various embodiments, only specific components or parts of the apparatus or system, for example the nano-projections 25, are made from biocompatible materials. A combination of different types of materials, for instance metallic and non-metallic materials, as well as biocompatible and non-biocompatible materials, can be used for manufacturing different components or elements of particular apparatuses or systems.

In particular embodiments of the present disclosure, the nano-projections 25 are made, or constructed, using a biocompatible and/or biodegradable polymer, for example Poly Lactic Acid (PLA), Poly Glycolic Acid (PGA) or Poly Lactide-co-Glycolide (PGLA). In embodiments wherein the nano-projections 25 are nano-rods 25a, the nano-rods 25a can be coated with at least one pharmaceutical composition (e.g., vaccine) of choice. In numerous embodiments, the nano-projections 25 are made, or constructed, using Zinc oxide (ZnO) or other metallic oxides.

In several embodiments, various components of the nano-projection array 20 and/or the application unit 100, for example the carrier substrate 30, can be made from material selected from a group including silicon, silicon oxynitride, tetraethylorthosilicate, wet silicon oxide, dry silicon oxide, chemical silicon oxide, silicon nitride, silicon carbide, gallium arsenide, aluminum oxide, silicaide, barium strontium titanate, lead zirconium tantalite, zinc oxide, organic material, metals, metal oxides, conductors, ceramics and polymers.

In some embodiments, the carrier substrate 30, for example the first layer 50a of the carrier substrate 30, can include, or be at least partially coated with, a material selected from a group including zinc oxide, silicon, silicon oxynitride, tetraethylorthosilicate, wet silicon oxide, dry silicon oxide, chemical silicon oxide, silicon nitride, silicon carbide, gallium arsenide, aluminum oxide, silicaide, barium strontium titanate, lead zirconium tantalite, organic material, metals, metal oxides, conductors, ceramics and polymers.

In particular embodiments, the carrier substrate 30 of the nano-projection array 20 and the displaceable carrier 120 of the application unit 100 are made, or constructed, using silicon. In particular embodiments, the silicon carrier substrate 30 and displaceable carrier 12 is nontoxic, biodegradable, and/or environmentally friendly.

Although particular material(s) are disclosed for the manufacture of particular apparatuses and systems, more specifically the various components or elements of the apparatuses and systems, of the present disclosure, other materials not disclosed herein can also be used within the scope of the present disclosure. In general, an apparatus or system in accordance with the present disclosure can be manufactured using micro-mechanical fabrication techniques and nano-fabrication techniques.

In order that the embodiments of the present disclosure may be more clearly understood as to matters of principle as well as to methods of construction and use thereof, several non-limiting examples are provided below. Reference numerals are not included for the examples that are provided below. In the examples described below, a reference made to particular apparatuses, systems, nano-projection arrays, application units, and various components or elements thereof, can be understood to include a reference to that which is described above and/or shown in FIG. 1A to FIG. 15B, or an equivalent or analog thereto. In addition, in the example described below, a reference made to particular apparatuses, systems, nano-projection arrays, application units, and various components or elements thereof, can be understood to include variants, and/or alternatives, to that which is described above and/or shown in FIG. 1A to FIG. 15B within the scope of the present disclosure.

### Example One: Method for Manufacturing a Nano-Rod Array

A method or process for manufacturing, fabricating, or synthesizing a nano-projection array wherein the nano-projections of the nano-projection array are nano-rods (i.e., nano-rod array) is provided in accordance with particular embodiments of the present disclosure.

### Formation or Preparation of Aligned Zinc Oxide (ZnO) Nano-Rods on a Silicon Carrier Substrate

In example one, the nano-rod array includes nano-rods made of Zinc oxide (ZnO) and a carrier substrate (or carrier medium) made of silicon. In a first process portion of the process of example one, a set of nano-rods are fabricated or manufactured on a silicon carrier substrate.

Vertically aligned Zinc Oxide nano-projection arrays or chips, more specifically nano-rod arrays or chips, can be synthesized following a procedure described in C. Li, G. Fang, Q. Fu, F. Su, G. Li, X. Wu, X. Zhao, Effect of substrate temperature on the growth and photoluminescence properties of vertically aligned ZnO nanostructures, Journal of Crystal Growth, 2006, 292, 19-25 (4).

The procedure uses a vapour-solid (VS) mechanism on Silicon wafers (i.e., silicon carrier substrates) that are coated with a Zinc Oxide seed layer of approximately 200 nm.

The above procedure generally induces, forms, or produces, perfectly, or near perfectly, vertical nano-rod arrays. Accordingly, to manufacture or form the conical, tapered, layered, stacked, and/or segmented nano-rods of the nano-rod array of example one, the procedure described in C. Li et al. is modified.

The reference of C. Li et al. only discloses the manufacture of single segment nano-rods. However, the nano-rods of the nano-rod array of example one are multi-segmented (i.e., include at least two segments). Accordingly, in the process or method of example one, each growth step is repeated a number of times, for instance four or more times, for forming the multi-segmented nano-rods. The procedure of each individual growth step can be analogous to or based upon that described in the above reference of C. Li et. al.

The novel use of multiple growth steps for forming multi-segmented nano-rods according to embodiments of the present disclosure, and provided in example one, enables production or formation of nano-rods with a stronger base and enhanced structural integrity.

In particular embodiments of the present disclosure, the growth of multi-segmented nano-rods is performed through a homoepitaxial anisotropic growth process on a set of already-grown ZnO nano-rods. The final number of segments of particular nano-rods can be dependent upon the total number of growth cycles of the homoepitaxial anisotropic growth process.

The multiple growth cycles of the present homoepitaxial anisotropic growth process can be achieved through pulsed laser deposition or magnetron sputtering, which is capable of producing vertically projecting, multi-segmented nano-projections (i.e., nano-rods or nano-wires).

The nano-rod array obtained using the above procedure has an average surface area of approximately 0.64 cm² (0.8 cm×0.8 cm), and includes ZnO pyramidal nano-rods with a tip size (or first end diameter) of approximately 60 nm and a length of between approximately 20 μm and 100 μm. The base size (or second end diameter) of the nano-rods is approximately 150 nm, while the distance between two adjacent nano-rods is approximately between 0.3 μm and 0.5 μm.

Adsorption or Coating of a Composition (e.g., Vaccine) onto the Nano-Rod Array

In a second process portion of example one, a composition, more specifically a vaccine or vaccine formulation, is adsorbed or coated onto the nano-rod array.

The vaccine, or vaccine formulation, of example one includes an antigen(s) capable of eliciting an immune response against a human pathogen. Examples of vaccines that can be used in association with the present disclosure (e.g., with the process of example one) are vaccines that include antigens capable of eliciting an immune response against human herpes viruses, hepatitis B virus (for example Hepatitis B Surface antigen), hepatitis A virus, or influenza virus antigen (H1N1).

In various embodiments of the present disclosure, for instance with the process of example one, lyophilized forms of vaccines, which are originally prepared as suspensions of one or more antigens together with excipients and stabilizers (e.g. alum, mannitol, dextran) are coated or adsorbed onto the nano-rod array.

The advantage derived from adsorption of a vaccine on solid nano-projections (i.e., nano-rods) includes reduced costs derived from avoiding the necessity of a cold chain. Many vaccines conventionally must be maintained between 2° C. and 8° C. (e.g. polio, varicella and yellow fever vaccines are sensitive to heat, while pertussis or hepatitis B vaccines are sensitive to freezing). The storage and/or delivery of vaccines, or vaccine formulations, in accordance with various embodiments of the present disclosure abolishes the necessity of a cold chain, which is particularly advantageous in developing countries.

For effectuating adsorption of the vaccine onto the surface of the nano-rods, 16 mg of albumin is weighed and mixed with 3 ml of Phosphate buffer Saline (PBS) in a 5 ml centrifuge tube. For in vivo studies, a higher initial concentration (21 mg) is used. The contents of the tube are sonicated for 2 minutes. 1 ml of albumin solution from the above tube is then poured into a well of 24-well plate. This is repeated three times, for a total of 3 ml divided into three wells. The tube with rest of the albumin solution was kept inside the freezer/fridge.

Three chips are deposited onto the three wells and the whole 24-well plate, having both albumin solution and chips, are kept inside the freezer/fridge for 24 hours. The albumin solution left in the well plate is then collected into three other tubes and quantified by Bradford analysis.

Quantification of Vaccine Adsorbed onto the Nano-Rod Array

A third process portion of the process of example one involves a quantification of vaccine adsorbed onto the nano-rod array.

A stock solution of 10 mg/ml of vaccine (e.g. OVA) in PBS is prepared. A total of 400 μg-1 mg of vaccine is applied and adsorbed onto the as-prepared nanorods, in the presence or not of 10-100 μl of adjuvant. Different concentrations (0.1-1.5 mg/ml) of vaccine are prepared by diluting a stock solution with different volume of PBS. 10 μl of each sample of said concentrations is added to separate wells of the 96-well plate. In addition, to the blank wells of the 96-well plate, 10 μl of a buffer solution are added. To each well being used, 200 μl of Bradford reagent (Sigma) are added and mixed. The 96-well plate is then incubated at room temperature for 15 minutes. Then Optical Density (OD) is taken at 610 nm for all the concentrations and a graph is plotted to get the standard curve.

Samples are exposed to OD measurements at 610 nm. This OD is then compared with the standard curve to get the appropriate protein concentration.

After the quantification of the quantity of vaccine adsorbed onto the nano-rod array, suitable nano-rod arrays (e.g., nano-rod arrays that includes at least a predetermined volume of vaccine) can be coupled to application units in preparation for use in the delivery or administration of the vaccine.

### Example Two: Method for Manufacturing a Nano-Needle Array

A method or process for fabricating or manufacturing a nano-projection array wherein the nano-projections are nano-needles (i.e., nano-needle array) is provided in accordance with particular embodiments of the present disclosure.

### Formation of Aligned Zinc Oxide (ZnO) Nano-Needles on a PDMS Carrier Substrate

A first process portion of example two involves the formation or preparation of Zinc oxide nano-needles on a silicon carrier substrate. FIG. 17A and FIG. 17B show different views of a number of zinc oxide nano-needles carried by a silicon carrier substrate.

The nano-needles that are formed on the silicon carrier substrate include a channel formed therethrough (i.e., the nano-needles have a hollow bore). The channel has a diameter in the nanometer scale. In many embodiments, the channel has a diameter that is smaller than approximately 1 μm. In example two, the nano-needles of the nano-needle array that is manufactured are separated by a distance of approximately 2 μm in order to achieve an array of at least 9×10⁶ nano-needles on a 1 cm² carrier substrate.

In a second process portion of the process of example two, the as-prepared aligned nano-needles on the silicon carrier substrate is treated with a still-liquid PDMS layer and subjected to a curing process at 90-120° C. in a hot-box oven for 10 minutes.

The second process portion results in formation of a carrier substrate with a silicon layer and a PDMS layer. FIG. 18A and FIG. 18B show different views of a number of zinc oxide nano-needles carried by a carrier substrate with a PDMS layer carried by a silicon layer.

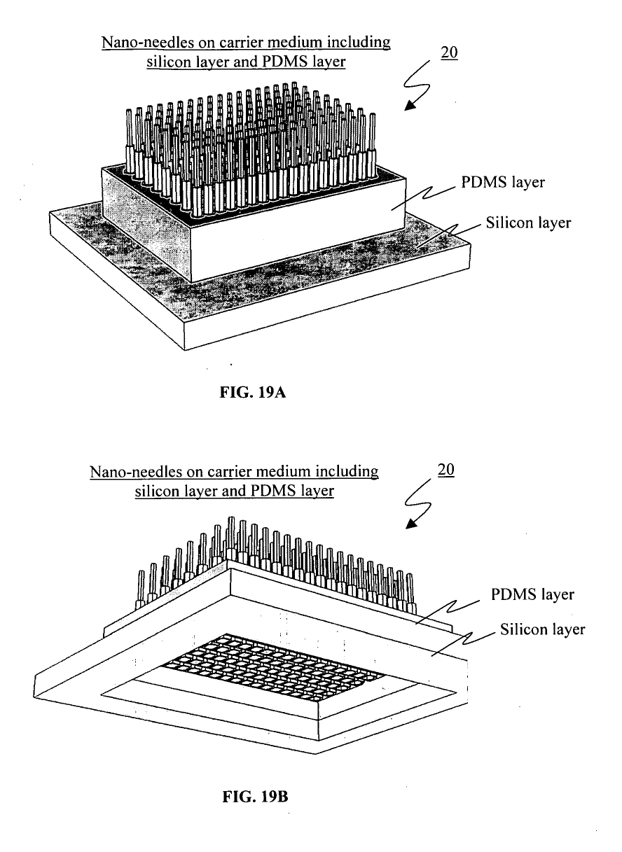

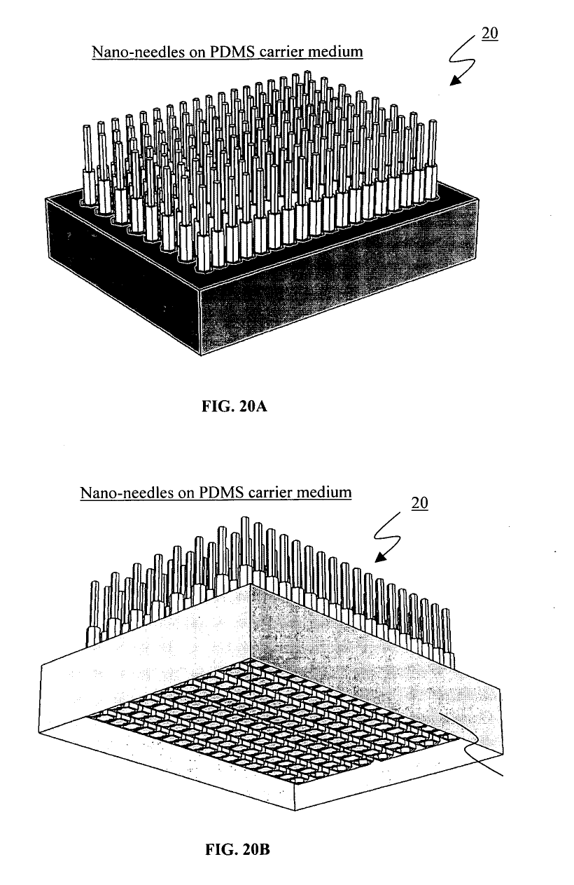

A third process portion involves treating the carrier substrate with Deep Reactive Ion Etching (DRIE) process to remove the silicon layer of the carrier substrate to thereby expose the PDMS layer with the nano-needles projecting therefrom. FIG. 19A and FIG. 19B show different views of a nano-needle array with nano-needles projecting from, and through, a PDMS layer.

### Providing a Volume of a Composition (e.g., Vaccine)

In a fourth process portion, a volume of a composition, more specifically a vaccine, is provided for use in association with the nano-needle array. As described above, a fluidic reservoir is used for storing or holding compositions (e.g., vaccines) to be delivered using nano-needles. The vaccine can be communicated from the fluidic reservoir through the channel of the nano-needles for delivery to the target site.

Although steps for further preparing the nano-needle array for use in delivering a vaccine are not described in example two, it will be understood such preparation steps can be similar to those of the process 300 as described above. For instance, the nano-needle array can be disposed within a peripheral structure and coupled to a displaceable carrier. The fluidic reservoir is disposed adjacent the displaceable carrier and the nano-needle array. A displacement of the displaceable carrier for inserting the nano-needles of the nano-needle array into the skin (e.g., to the epidermis) triggers, facilitates, and/or effectuates communication of the vaccine through the channels of the nano-needles for delivery at the target site (e.g., at the epidermis).

In particular embodiments of the present disclosure, for instance in example two, the nano-needles are used to deliver adenovirus-vectored vaccines which can be stored for long periods without the need for freezing [Evans Ru K, Nawrocki D K, Isopi L A et al. Development of stable liquid formulations for adenovirus-based vaccines. J. pharm. Sci., 2004, 93, 2458-2475]. In specific embodiments, the nano-needle array can also be used to deliver more viscous suspensions/gels containing anti-ageing substances and anti-cancer agents.

### Example Three: Integrity of the Nano-Projections (e.g., Nano-Rods) Carried by the Carrier Substrate

In example three, the integrity of the nano-projections, more specifically nano-rods, carried by a carrier substrate was studied or observed using scanning electron microscopy (SEM). More specifically, the nano-rods were visualized using SEM before and after insertion or penetration into the skin in order to analyze the integrity of the nano-rods carried by the carrier substrate before and after said insertion.

### Results

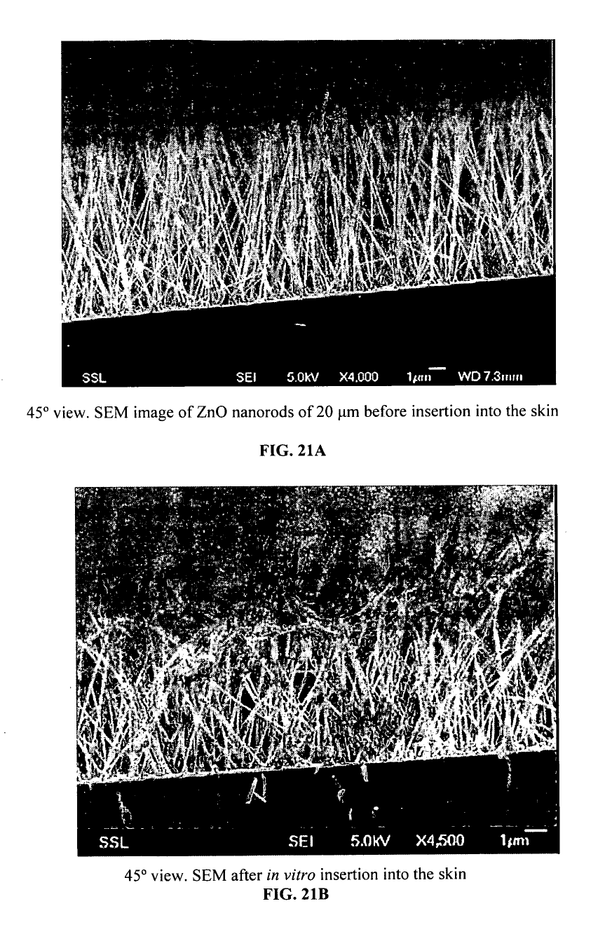

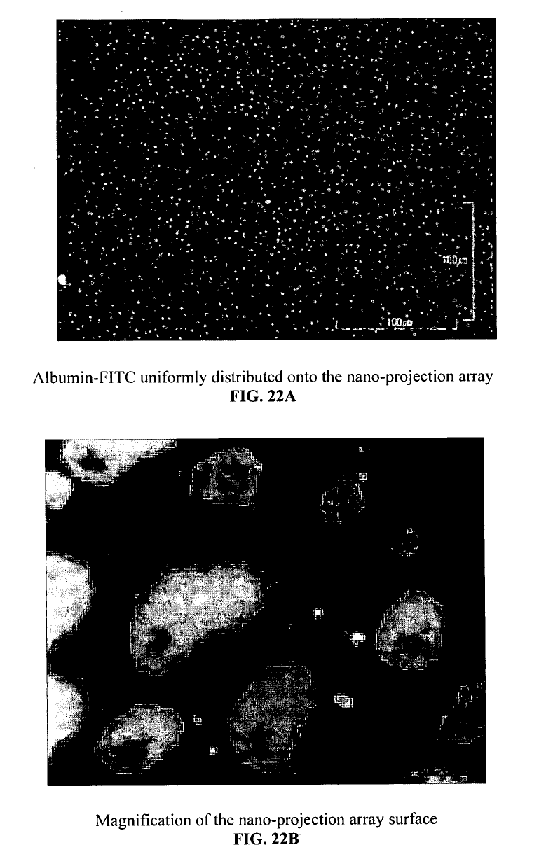

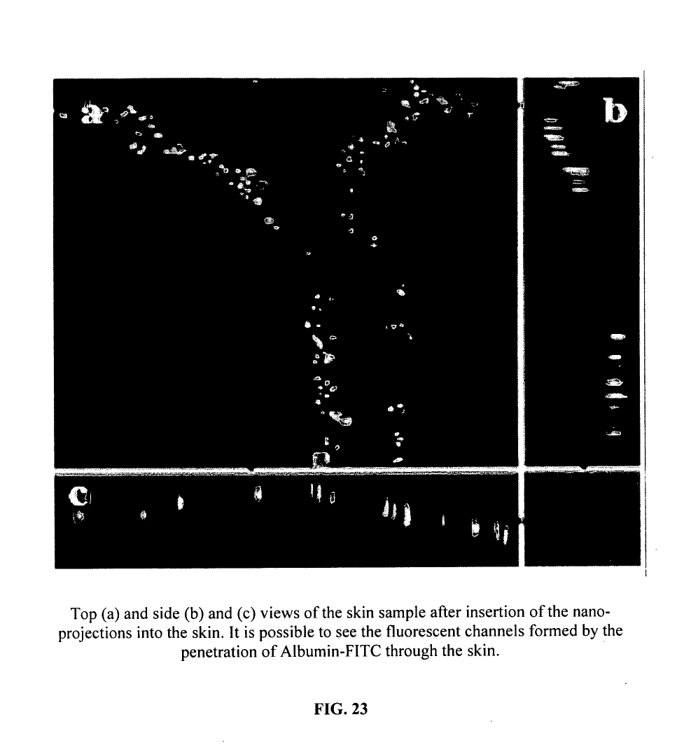

FIG. 21A shows the nano-projections (i.e., nano-rods) before insertion into the skin penetration and FIG. 21B shows the nano-projections (i.e., nano-rods) after insertion into the skin.

### Discussion of Results

Although FIG. 21A and FIG. 21B indicate a change in the shape of the nano-rods, it is important to note that the nano-rods remained attached to the carrier substrate, since no reduction in their density was noticed all along the sample. In addition, most of the nano-rods preserved a certain degree of alignment and only the tips were affected, thus indicating that the nano-rod array can potentially be used twice. The ability to recycle or reuse the same nano-rod array for delivering compositions (e.g., vaccines) enables the use of said nano-rod array to be more cost effective.

The change in the shape of the nano-rods tips is likely due to the pressure exerted for the effective application of the nano-rod array on the skin sample. The initial perforation of the skin, the subsequent penetration through the stratum corneum (at least 10 μm below the epidermis) and the successful delivery of the vaccine ideally require vertically aligned or slightly oblique nano-rods. The ability of the nano-rod array, more specifically the ZnO nano-rods of the nano-rod array, to maintain their structural integrity (or shape) facilitates confidence in delivering an intended composition dose and allows the nano-rod array to be used multiple times.

### Example Four: In Vitro Skin Penetration or Insertion Studies

In-vitro experiments (i.e., in vitro skin penetration studies) were conducted to study the degree of penetration or insertion of the nano-projections to the target site for subsequent absorption or delivery of the composition to the target site.

The skin penetration studies using Albumin-FITC were conducted across the stratum corneum of excised abdominal skin samples of a 22-year-old female Indian from the Singapore General Hospital (SGH), with the prior consent of the donor. Albumin-FITC, a protein conjugated with a fluorescent molecule, represented the vaccine prototype, offering the advantage to be further characterizable under the fluorescence microscope as shown FITC provided optical signals for the visualization of the vaccine-nano-projection array complex (as shown in FIGS. 22A and 22B).

Stratum corneum was isolated by immersing the whole skin sample in 60° C. water for 2 minutes, followed by careful removal of the stratum corneum from the connective tissues (5). Samples were stored in plastic bags at −80° C. until use. Prior to experiments, these membranes with the top of the stratum corneum sides up were floated over PBS. The Albumin-FITC was delivered through a nano-projection array that includes Zinc Oxide nano-projections (i.e., nano-rods). The stratum corneum (SC) sample was analyzed under Fluorescence Microscopy (as shown in FIG. 23). Franz flow-through type diffusion cells were used for the in vitro skin penetration studies of example four (5).

The nano-projection array was mounted, or applied, onto the isolated human stratum corneum (SC). More specifically, the nano-projections of the nano-projection array were facing the SC for insertion into the SC. A receptor compartment was coupled to the nano-projection array, and correspondingly, the SC. A receptor solution of 500 ml of PBS was placed in a reservoir bottle and allowed to flow into and through the receptor compartment at 0.50 ml/h. The receptor solution was thoroughly degassed to prevent the formation of bubbles beneath the membrane. Ambient temperature of the cells was controlled at 37° C. by a heater/circulator (Haake, Germany). The receptor solution was pumped by a 16-channel peristaltic cassette pump (Ismatec, Switzerland) continuously through the receptor compartment and drained into a test-tube sitting in the fraction collector (ISCO Retriever IV, US). Cumulated receptor liquid samples were taken at 4-hour intervals for protein assay. Experiments were carried out in triplicate, performed in different times over a period of 4 months.

Fluorescence and Confocal Laser Scanning Microscopy on the skin samples revealed that majority of the Albumin-FITC was absorbed within the channels inside the SC, proving that transdermal delivery by means of the nano-projection array was feasible. The detailed evaluation of the skin under the confocal microscopy demonstrated that the penetration was indeed facilitated by the presence of the nano-projections, more specifically nano-rods, as indicated by the formation of fluorescent pathways along the skin layer in a manner perfectly correspondent to the nano-channels that are created by the nano-projections, more specifically, nano-rods.

In other words, the fluorescent molecule was mainly absorbed through these nano-channels created by insertion of the nano-projections into the SC. The florescent signals (i.e., representing fluorescent molecules) were more stacked at the skin surface in between two consecutive nano-projections. In addition, no fluorescence or florescent signal was detected outside the area covered by the nano-projection array, thus confirming the utility of the nano-projection array to improve drug delivery, for instance, through the use of the nano-projection array to selectively deliver a drug (or other composition).

### Quantitative and Qualitative Protein Assay

The fluid passed through the skin during the skin penetration studies was subjected to Bradford protein quantitative assay (6) and SDS-PAGE (7) in order to confirm the eventual presence of Albumin-FITC. The experiments or studies of example four were performed in triplicate.

### Bradford Protein Quantitative Assay

Both the protein solutions (i.e., the protein solution before adsorption and the protein solution after adsorption on the nano-projection array) were subjected to Bradford protein assay to determine the amount of protein (i.e., composition) that was adsorbed onto the nano-projection array. The amount or quantity of protein was calculated using the difference from an initial concentration and an amount collected after the functionalization of the nano-projection array.

The table below (i.e., Table 1) shows that about 427 μg of Albumin-FITC was adsorbed on to the nano-projection array. In various examples described herein, a nano-projection array is also known as a nano-projection array chip or simply a chip. Out of 0.427 mg of available protein, only approximately 57 μg (about 13%) of the protein (i.e., composition) was delivered through the skin sample. A same protein solution can be used to charge several nano-projection arrays (i.e., one protein solution can be used with multiple nano-projection arrays) to facilitate minimal wastage of protein (i.e., composition) and hence facilitate an increased cost-effectiveness of the processes of the present disclosure.

TABLE 1
Protein concentration (mg/ml) present in particular protein samples

| | | | Protein conc. |
|---|---|---|---|
| Protein sample | OD at 595 nm | Average OD | (mg/ml) |
| Protein sample | 0.9486 | 0.9498 | 5.397 |
| before adsorption | 0.9463 | | |
| on to chip | 0.9545 | | |
| Protein sample | 0.8012 | 0.7987 | 4.544 |
| after adsorption | 0.7982 | | |
| on to chip | 0.7967 | | |
| Protein on chip | We used only | | 0.853/2 = |
| | 500 μl of the | | 0.427 |
| | protein solution | | |
| Protein sample | 0.0431 | 0.0457 | 0.302 |
| from washing of | 0.0461 | | |
| chip | 0.0479 | | |
| Protein sample | 0.0029 | 0.0023 | 0.057 |
| collected from | 0.0022 | | |
| receptor chamber | 0.0018 | | |

### SDS-PAGE

Finally, SDS-PAGE using each of the above protein samples was performed. Results from SDS-PAGE showed a band in the gel that corresponds to the molecular weight (66 kDa) of the protein (i.e., composition) thus confirming the penetration of Albumin-FITC through the skin layer.

### Example Five: In Vivo Skin Penetration or Insertion Studies

In vivo experiments or studies were conducted to study the degree of penetration or insertion of the nano-projections to the target site for subsequent absorption or delivery of the composition to or at the target site.

Skin penetration experiments were conducted on three nude mice. Female BALB/c mice, of 6-8 weeks of age at the beginning of each experiment, were obtained from National Laboratory Animal Center, Mahidol University, Thailand. The mice were kept under standardized condition at the animal facility of the Faculty of Pharmaceutical Sciences, Naresuan University. They had free access to rodent chow and water and they were used in accordance with the guidelines of the National Research Council, Thailand. Each mouse was carefully shaved on dorsum 24 hours to 48 hours prior to immunization study.

### Sample Collection

Blood and fecal samples were collected on days 0 (before immunization) and 35 (at the end of study). On day 0, blood sample's (volume of 0.2 ml per animal) were collected from the cut tail tip of mice. However, at the end of the study (day 35), the blood samples (volume of 0.2 ml per animal) were collected by cardiac puncture following anesthesia of the mice with diethyl ether. The blood samples were allowed to clot overnight and then centrifuge at 8,000 g for 5 minutes at room temperature. For both tail bleeds and cardiac puncture, serum was collected for each mouse and kept separately. All serum samples were stored frozen at −20° C. until assayed. Fresh fecal samples from mice were collected at the same time as blood samples. The samples were kept at −20° C. Before assayed the samples were subjected to vacuum drying using a Speed Vac concentrator (LABCONCO, Missouri, USA).

### Quantitative and Qualitative Protein Assay

The fluid passed through the skin during skin penetration studies was subjected to Bradford protein quantitative assay (6) and SDS-PAGE (7) in order to confirm the eventual presence of Albumin-FITC.

### Bradford Protein Quantitative Assay

A comparison with the samples of the in vitro studies and the samples prepared for the in vivo studies (in which the initial concentration was much higher (7.107 mg/ml) in order to maximize the amount of vaccine adsorbed onto, and delivered from, the nano-projection arrays (also referred to as chips)) was performed.

No significant difference was observed between the samples obtained from the in vitro studies and the samples obtained from the in vivo studies, suggesting that saturation can be achieved with lower composition concentrations. In other words, the amount of protein (or composition) that could be physically adsorbed on the nano-projection arrays is in a range of between approximately 427 μg and 503 μg.

In the case of in vivo studies, the nano-projection arrays were treated with OVA Albumin. As indicated by Table 2 as shown below, the most of the protein (more than 70%) was released from the nano-projection arrays and diffused through the skin, indicating a successful release of vaccine prototype (i.e., delivery of vaccine).

Another important aspect to note is that, even though the nano-projection arrays of example five were manually fabricated and cut, the nano-projection arrays showed reproducible characteristics, as indicated by their uniform appearance under SEM and the amount of protein the nano-projection arrays adsorbed (range of approximately 450 μg to 503 μg of Albumin/chip). This last aspect represents an important result for scalability of the above-described procedure.

Amount of protein adsorbed on to the nano-projection array (or chip) for in vivo skin permeation studies was quantified in the experiments of example five. More specifically, the protein solution before and after adsorption onto the nano-projection array (or chip) was analyzed by Bradford assay.

The “x” in table 2 below indicates that the nano-projection array 2 (or chip 2) that was used for analysis under SEM, for which therefore it was not possible to calculate the quantity of protein (i.e., composition) that remained onto the nano-projection array (or chip).

TABLE 2
Protein concentration (mg/ml) present in particular protein samples

| | | | | Protein conc. | |
|---|---|---|---|---|---|
| Protein sample | OD at 595 nm | | Average OD | (mg/ml) | |
| Protein sample | 1.12615 | | | 7. 107 | (stock |
| before adsorption | | | | | solution) |
| on to chip | | | | | |
| Protein Sample | 1.1362 | (chip 1) | | 6.401 | (chip 1) |
| after adsorption | 1.0355 | (chip 2) | | 5.834 | (chip 2) |
| on to the chip | 1.1713 | (chip 3) | | 6.599 | (chip 3) |
| Protein on chip | | | | 0.706 | (chip 1) |
| | | | | 1.273 | (chip 2) |
| | | | | 0.508 | (chip 3) |
| Protein sample | 0.03603 | (chip 1) | | 0.203 | (chip 1) |
| from washing of | x | (chip 2) | | x | (chip 2) |
| chip | 0.01011 | (chip 3) | | 0.0569 | (chip 3) |
| Protein delivered | | | | 0.503 | (chip 1) |
| through the skin | | | | x | (chip 2) |
| | | | | 0.451 | (chip 3) |

### SDS-PAGE

Finally, SDS-PAGE using each of the above protein samples was performed. Results from SDS-PAGE showed a band in the gel that corresponds to the molecular weight (66 kDa) of the protein (i.e., composition) thus confirming the penetration of Albumin-FITC through the skin layer.

### Example Six: Tape Stripping Experiment

Experiments (i.e., tape-stripping experiments) were conducted to demonstrate or study the profile of distribution of the composition when delivered into the skin. In other words, tape-stripping experiments were conducted to demonstrate or study whether the composition or protein delivered by the nano-projection array was able to effectively penetrate through the skin for delivery to the target site.

In the experiments of example six, tape strips were removed from the skin area after a one-hour application of one nano-projection array (or chip) that had been previously adsorbed with Albumin-FITC until a shiny watery layer appeared (wet epidermis layer). The application of the nano-projection array (or chip) was onto the skin of the dorsal part of the forearm of a 65-year old volunteer for a period of one hour.

The adhesive tape (“Transpore tape” from 3M Company) was pressed onto the surface of the skin and removed with one quick movement and subsequently fixed directly to a slide frame. The above-described tape stripping procedure was repeated 18 times on the same skin area, and the correspondent slide frames were analyzed under fluorescence microscope.

### Results and Discussion of the Results

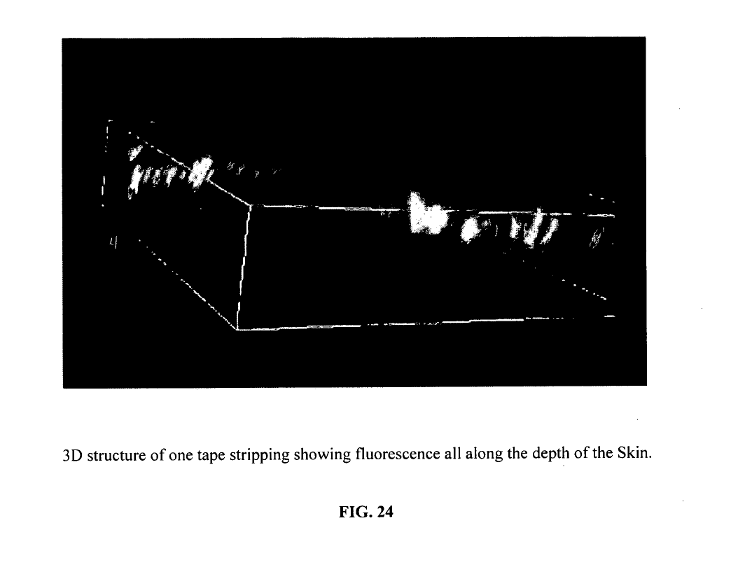

FIG. 24 shows an analysis under fluorescence microscope of the collected strip layers revealed the presence of fluorescence signals all along the strip layers, from a first strip layer to a last strip layer. Results shown in FIG. 24 suggest that, even though the nano-projections (e.g., nano-rods) are approximately 20 μm in length, the nano-projections are sufficiently strong and aligned to create nano-holes into the skin to facilitate or effectuate the delivery of the composition (e.g., vaccine) into the skin.

### Example Seven: Trans-Epidermal Water Loss

Experiments were conducted to study or measure the skin's modification due to insertion of the nano-projections into the skin. More specifically, experiments were conducted to study the effect on trans-epidermal water loss due to the insertion of the nano-projections into the skin.

The Trans-epidermal Water Loss (TEWL) technique is widely used since it is a quick and non-invasive biophysical technique (8,9). TEWL is defined as the measurement of the quantity of water that passes from inside a body through the epidermal layer (skin) to the surrounding atmosphere via diffusion and evaporation processes. Such measurement can be useful to determine skin damage or, in a specific case, to evaluate the nano-rod-mediated-enhancement of skin permeation.

In the experiments of example seven, the TEWL before and after insertion of the nano-projections into the skin was measured with a Tewameter, which consisted of a closed transparent chamber containing sensors for relative humidity and temperature.

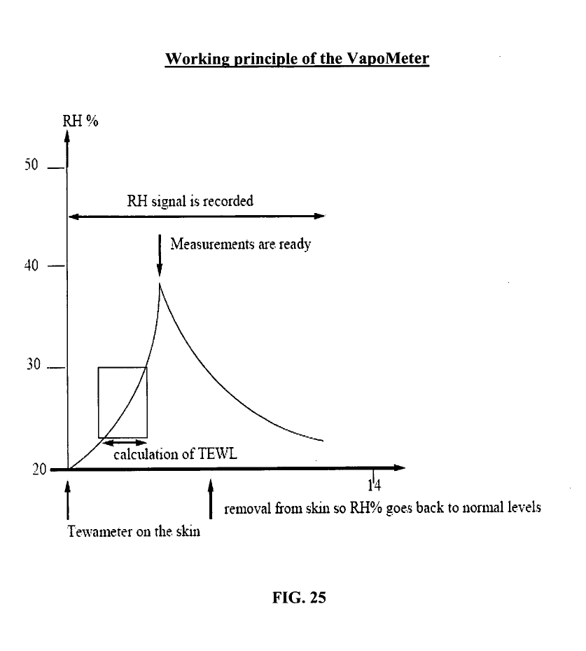

FIG. 25 illustrates the working principle of a vapometer used for measuring or evaluating the TEWL. FIG. 25 shows a linear increase of relative humidity (RH %) in the chamber shortly after placing a nano-projection array in contact with the skin (i.e., inserting the nano-projections of the nano-projection array into the skin). The TEWL is calculated from the increase in RH %.

The study was conducted in the early afternoon (13:30) in a temperature controlled room (temperature was kept at 21° C.) and the subject acclimatized for about 20 minutes prior to the start of the experiment. Four angles of a squared area were marked on each forearm in the center of which the TEWL were measured before and after the nano-projections were inserted into the skin. Before inserting the nano-projections into the skin, baseline values were recorded for the barrier function (TEWL). TEWL was measured using Tewameter™ 300 Courage+Khazaka (Cologne, Germany) at the Cosmetic and Natural Product Research Centre (Cosnat), Naresuan Hospital, (Phitsanulok, Thailand). The probe of the apparatus was placed gently onto the center of a marked square on the skin, and values were collected over a period of 30 seconds, after which an average reading was automatically generated.

Measurements on the two arms were performed at different time. Baseline measurements were made every 5 minutes for 20 minutes. The values were expressed as g h⁻¹m⁻² and were calculated from a mean of three consecutive measurements. The measurements were performed directly after a two-fold application (i.e., two-fold insertion of nano-projections into the skin), at 0 minutes and repeated every 5 minutes over a period of 25 minutes.

### Results and Discussion

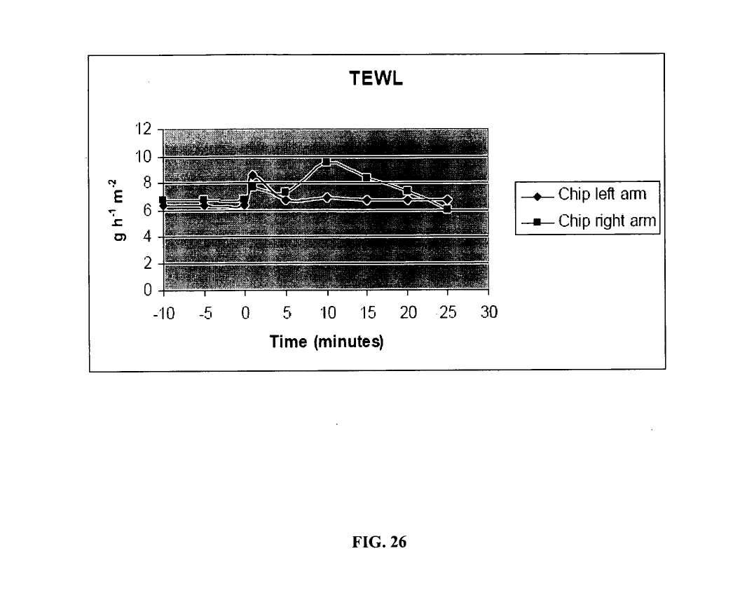

The TEWL values after treatment with the devices are provided in the graph of FIG. 26.

With regard to the left arm, prior to treatment (i.e., prior to insertion of the nano-projections into the skin) the TEWL values were about 6.30 g h⁻¹m⁻² (SD±0.7). After the application of the nano-projection array (or chip) onto the skin (i.e., insertion of the nano-projections into the skin), the TEWL values increased immediately above 8.57.

After 5 minutes of rest, the following TEWL measurement provided already a much lower value (≦7.00), which decreased rapidly in subsequent measurements. The measurements were continued in order to cover a whole period of 70 minutes (data not shown), but the values did not change remarkably after the initial 15 minutes to 25 minutes. Therefore, further points in the final graph are not included.

In the case of the right arm (pink line), a different behavior was noticed, with baseline levels of about 6.68 g h⁻¹m⁻² (SD±0.7). With the right arm, the increase in TEWL was comparable, but this occurred only after 10 minutes from the application, and the effects of the nano-projection array application decreased slowly until baseline values were reached after about 15 minutes.

An explanation for the difference between the left and right arms could be attributed to the temperature inside the room, which at the beginning seemed pleasant but subsequently resulted in a temperature that was too cold for the subject under investigation. It has been already demonstrated that TEWL is affected by environmental factors such as humidity, temperature, time of year (season variation) and moisture content of the skin (hydration level). While the chamber of the apparatus controlled the humidity, the ambient temperature on the individual was maintained constantly at 21° C. but it was perceived as uncomfortable.

In every case, it was important to observe an increase in permeability and an enhancement of water loss values (TEWL in both arms increased of at least 2.3 g h⁻¹m⁻²) with the use of the chips, as a demonstration of their efficacy in penetrating the skin with a total absence of pain. Therefore, they seemed to be able to disrupt the stratum corneum barrier to a high extent. However, the penetration of the nano-projections into the skin induced no irritation on the treated area. In addition, the subsequent decrease of TEWL values to normal (or base) ranges suggested that the nano-holes or nano-openings that are formed due to the insertion of the nano-projections into the skin rapidly closes after a removal of the nano-projections from the skin, thus minimizing both skin irritation and any prolonged adverse effect.

The in vivo experiments measuring the TEWL showed that an increased water flux was obtained after application of the nano-projection array (i.e., insertion of the nano-projections into the skin) and that the viable epidermis had been reached, or reproduced, with the short nanoneedles with a length of about only 20 micrometers.

The experiment also showed that after only 40 minutes after removal of the chip, the TEWL was reduced again to the normal (or base) value of the untreated skin indicating that the nano-pores or nano-holes created across the stratum corneum (due to insertion of the nano-projections into the skin) are closed again after a short time. The rapid closure of the nano-pores or nano-holes formed within the skin can prevent micro-organisms from entering or penetrating the skin via said nano-pores or nano-holes.

Therefore, experiments of example seven indicate that apparatuses, devices, systems, methods, and processes provided by various embodiments of the present disclosure can have unique and advantageous properties regarding safety and efficacy which are not fulfilled by currently existing micro-needle systems or other devices used for delivering pharmaceutical or chemical compositions (e.g., vaccines).

### Example Eight: Determination of Immune Responses

Experiments were conducted to demonstrate or evaluate the ability of particular nano-projection arrays of the present disclosure to deliver a composition, more specifically a vaccine, for evoking an immune response.

To demonstrate or evaluate the ability of the nano-projection arrays to deliver an effective dosage of a composition, more specifically a vaccine, to evoke an immune reaction, an immune reaction test was performed using BALB/c mice.

Immune responses to ovalbumin (OVA) were analyzed after 5 weeks by Enzyme-Linked Immunosorbent Assay (ELISA) in order to determine the levels of OVA-specific serum immunoglobulin G (IgG) antibody as described by Pitaksuteepong (10). The flat bottoms of the wells of 96-well MaxiSorp NUNC-Immuno™ plates were coated with 500 per well of 100 μg/ml OVA in coating solution (0.1 M NaHCO₃, pH 8.2). After an overnight incubation at 4° C., the plates were washed six times with 0.05% v/v Tween 20 in phosphate buffer saline solution (T20/PBS). Blocking was carried out by adding 200 μl of 10% v/v FBS in PBS (10 FBS/PBS) into the wells followed by a two hour incubation at room temperature. The plates were then washed with T20/PBS. Subsequently, 100 μl of serum was added to each well in duplicate. Two fold serial dilutions of samples with 10 FBS/PBS were carried out in the ELISA plates. Blanks were also set up in duplicate using 100 μl of 10 FBS/PBS. Absorbance values of these blanks will be subtracted from the absorbance values of the standards and samples.

The ELISA plates were incubated for one hour at room temperature and then washed with T20/PBS. Goat anti-mouse IgG HRP conjugates were diluted with 10 FBS/PBS and 100 μl of the resultant diluted solution was added into each well. The plates were then further incubated for 45 minutes at room temperature. Subsequently, the plates were washed with T20/PBS and 100 μl of TMB, which were added into each well. After the color development, the reaction was stopped by adding 1000/well of 1N H₂SO₄. The absorbance of each well was measured at a wavelength of 450 nm using a microplate reader (Spectra count, Perkin Elmer, USA).

The specific IgG antibody titers were determined after 5 weeks of incubating in the three mice. The zero value was defined as the mean of all data, which showed no significant serum concentration dependence. It is important to note that IgG values obtained at each time frame (i.e., at day 0 and at day 35) showed good uniformity (i.e., small standard deviation) within each group of mice. Hence, the results indicate a good reproducibility of the protocol that was used.

### Results and Discussion

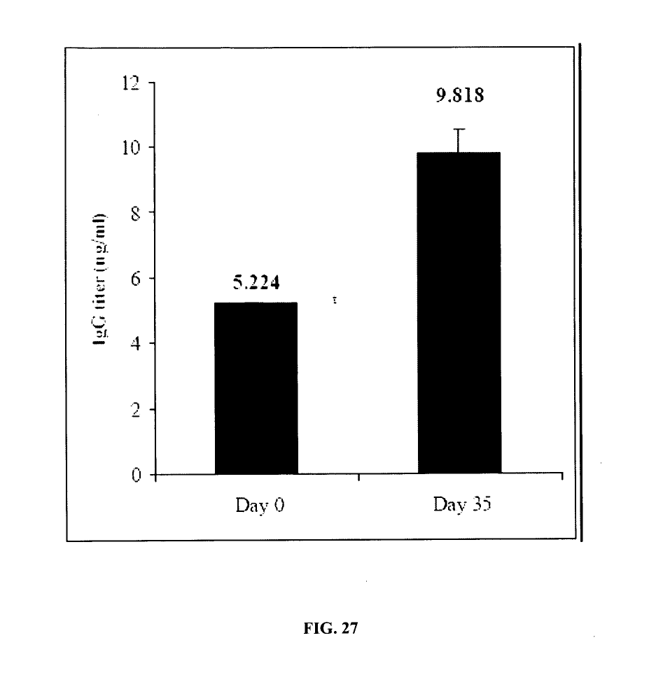

As reported on Table 3 provided below and in the graph of FIG. 27, the resulting improvement of immune response produced (or immunity provided) was about 50%. In other words, the administration of vaccine using an apparatus of the present disclosure causes an approximately 50% improvement in the immune response produced. In addition, the weak immunogenic properties of OVA (11) may have limited or reduced the improvement in the immune response produced. This is because the vaccine prototype that was used with the experiments of example eight is usually associated to suitable adjuvants like chitosan and trimethyl chitosan (TMC) (12) in immunogenic studies. Accordingly, a higher percentage of improvement of immune response produced can be expected using apparatuses of the present disclosure with improved vaccine prototypes or altered vaccine formulations.

TABLE 3
IgG concentration (ng/ml) at each of Day 0 and Day 35

| Day | IgG concentraiton (ng/ml) | | S.E. |
|---|---|---|---|
| Day 0 | | 5.224 | — |
| Day 35 | | 9.818 | 0.696584 |
| | Chip 1 | 10.962 | |
| | Chip 2 | 9.936 | |
| | Chip 3 | 8.558 | |

It is widely believed that the Langerhans cells in the skin, as antigen-presenting cells, play a decisive role in the process of cutaneous antigen processing and presentation (13-15). However, Langerhans cells are only very efficient in processing intact proteins, but are relatively poor presenters of their fragments. In general, antigen fragments are presented differently from the large epitopes (14). It has even been postulated that Langerhans cells trap antigens in the epidermis and carry them to the draining lymph nodes where the corresponding fragments could be eventually presented to T cells (13). It could therefore also be possible that the original vaccine was modified and reduced into smaller fragments during the experiment. Hence, these may be explanations for the modest antigen titers found in the first pilot vaccination study.

The amount of OVA necessary to induce proper immunization in mice is about 100 μg. On the basis of previous results from tape stripping (as shown in FIG. 24), in which fluorescence was observed until the deepest layer of the stratum corneum) and on the calculation of antigen released from the nano-projection array (or chip) (i.e., 450-503 μg of Albumin per chip, as indicated in Table 2 provided above), one can deduce that at least ⅕ of the whole dose has reached the desired target site.

### Example Nine: A System Including Multiple Nano-Needle Arrays and a Corresponding Number of Fluidic Reservoirs

A system that includes multiple, for instance two, three, four, five, ten, or more, nano-needle arrays and a corresponding number of fluidic reservoirs is provided in accordance with an embodiment of the present disclosure, and described in example nine. The system is configured to deliver a composition, more specifically a vaccine, to the epidermis (epidermal layer) of the skin of an organism.

Each nano-needle array includes a set of nano-needles, each nano-needle including a channel formed therethrough, and a carrier medium configured to carry the set of nano-needles. The nano-needles project from the carrier medium in a perpendicular, or substantially perpendicular, manner relative to a surface of the carrier medium. The nano-needles have a conical, tapered, layered, and segmented shape, which can enhance the structural integrity of the nano-needles.

Each nano-needle array can be received or disposed within a peripheral structure or housing. In addition, each nano-needle array is carried on a displaceable carrier. The displaceable carrier is coupled to the peripheral structure and is configured to be displaced between a first position (or retracted position) and a second position (or extended position) relative to the peripheral structure. The displaceable carrier is configured to be biased at the first position. Accordingly, the displaceable carrier is configured to return to the first position subsequent a displacement thereof to or toward the second position.

The displacement or position of the displaceable carrier at the first position correspondingly positions the set of nano-needles of the nano-needle array within the peripheral structure. The displacement or position of the displaceable carrier at the second position correspondingly positions the set of nano-needles of the nano-needle array outside the peripheral structure for enabling insertion of the set of nano-needles can into the skin of an organism.

The distance between the first position and the second position can be controlled, for example selected and varied, for enabling displacement of the set of nano-needles preferentially to the epidermis of the skin. The distance between the first position and the second position, and hence displacement of the displaceable carrier, is controlled using a displacement control element. The displacement control element is disposed and configured relative the displaceable carrier to limit the displacement of the displaceable carrier to between the first and second positions. The displacement control element of example nine is a set of tensioned elements, for instance springs, which are coupled to the displaceable carrier. However, it will be understood that other deformable structures or units, and/or rigid structures or units, can be used for controlling the displacement of displaceable carrier.

In the system of example nine, each nano-needle array is associated with a fluidic reservoir. The channels of the set of nano-needles of each nano-needle array are fluidly communicable with the corresponding fluidic reservoir. Each fluidic reservoir is shaped and configured to store a volume of a specified vaccine. For example, with the system of example nine, a first fluidic reservoir stores a diphtheria antigen vaccine and a second fluidic reservoir stores a tetanus antigen vaccine. It will be understood that other types of vaccines, for example adenovirus-vectored vaccines, can be stored within the fluidic reservoirs.

The displacement of the displaceable carriers of the system of example nine to or toward the second position (i.e., extended position) enables insertion of the sets of nano-needles of the multiple nano-needle arrays into the skin, and more specifically to the epidermis of the skin. In addition, the displacement of the displaceable carrier triggers, facilitates, or effectuates communication of the vaccines stored within the fluidic reservoirs of the system through the channels of the corresponding sets of nano-needles for delivery to the target site, more specifically epidermis of the skin.

As described above, embodiments of the present disclosure relate to systems, apparatuses, devices, methods, and processes that involve nano-sized projections to administer or deliver a biological, pharmaceutical, or chemical composition, for example a vaccine, to a target site. In most embodiments, the nano-sized projections can be inserted to a predictable and/or controllable depth within the epidermis of a body.

The apparatus or device of many embodiments include a nano-projection array that includes a set of nano-projections that is carried or supported by a carrier substrate or a carrier medium, and an application unit that is configured to facilitate or effectuate displacement of the nano-projection array. In most embodiments, the application unit includes a peripheral structure or peripheral housing within which the nano-projection array can be disposed or positioned. In addition, the application unit includes a displaceable carrier that is couplable to the nano-projection array. The displaceable carrier can be displaced relative to the peripheral structure to thereby displace the nano-projection array that is coupled to, carried by, or associated with the displaceable carrier.

More specifically, the displaceable carrier can be displaced between a retracted position to an extended position to thereby displace the nano-projection array between corresponding retracted and extended positions. In several embodiments of the present disclosure, the displacement of the displaceable carrier is controlled or limited. For instance, the distance of the displacement of the displaceable carrier can be controlled or limited. Displacement of the nano-projection array to the extended position facilitates or effectuates insertion of the nano-projections of the nano-projection array into the body, for instance to the epidermis within the skin. In several embodiments, a set of force management elements can be used to control the insertion of the nano-projections into the body.

Aspects of particular embodiments of the present disclosure address at least one aspect, problem, limitation, and/or disadvantage associated with existing apparatuses, systems, and methods for delivering pharmaceutical or chemical compositions into the body. While features, aspects, and/or advantages associated with certain embodiments have been described in the disclosure, other embodiments may also exhibit such features, aspects, and/or advantages, and not all embodiments need necessarily exhibit such features, aspects, and/or advantages to fall within the scope of the disclosure. It will be appreciated by a person of ordinary skill in the art that several of the above-disclosed systems, components, processes, or alternatives thereof, may be desirably combined into other different systems, components, processes, and/or applications. In addition, various modifications, alterations, and/or improvements may be made to various embodiments that are disclosed by a person of ordinary skill in the art within the scope and spirit of the present disclosure.

### REFERENCES

(1) L. Baizer, C. Lacey, J. Hayes, L. D'Antonio. Needle-free Injectors: new Technologies, The Drug Delivery Report, February 2001 PharmaVentures Ltd 2001.
(2) G. Furness. Nasal drug delivery: rapid onset via a convenient route. Published by ONdrug Delivery Ltd 2005, Cedar Cottage, Newtimber Place Lane, Newtimber, West Sussex, BN6 9BU, United Kingdom. Registered in England: No 05314696.
(3) D. P. Wermeling, S. L. Banks, D. A Hudson, H. S. Gill, J. Gupta, M. R. Prausnitz, A. L. Stinchcomb. Microneedles permit transdermal delivery of a skin-impermeant medication to humans. Proc. Natl. Acad. Sci. USA. 2008, 105(6), 2058-2063.
(4) C. Li, G. Fang, Q. Fu, F. Su, G. Li, X. Wu, X. Zhao. Effect of substrate temperature on the growth and photoluminescence properties of vertically aligned ZnO nanostructures. J. Cryst. Growth, 2006, 292, 19-25.
(5) A. M. Kligman, E. Christophers. Preparation of Isolated Sheets of Human Stratum Corneum. Archives of Dermatology., 1963, 88, 702-705.
(6) M. M. Bradford. A rapid and sensitive method for the quantitation of microgram quantities of protein utilizing the principle of protein-dye binding. Anal. Biochem., 1976, 72, 248.
(7) J. Sambrook et al. Molecular cloning: a laboratory manual, 20d ed. Cold Spring Harbor Laboratory Press, Cold Spring Harbor, N.Y., 1998.
(8) J. Pinnagoda, R. A. Tupker, T. Agner, J. Serup. Guidelines for transepidermal water-loss (TEWL) measurement—a report from the Standardization Group of the European Society of contact dermatitis. Contact Dermatitis, 1990, 3, 164-178.
(9). J. W. Fluhr, O. Kuss, T. Diepgen, S. Lazzerini, A. Pelosi, M. Gloor, E. Berardesca. Testing for irritation with a multifactorial approach: comparison of eight non-invasive measuring techniques
(10) S. Saraf, D. Mishra, A. Asthana, R. Jain, S. Singh and N. K. Jain. Lipid microparticles for mucosal immunization against hepatitis B. Vaccine, 2006, 24, 45-56.
(11) E. C. Lavelle, G. Grant, A. Pusztai, U. Miler, D. T. O'Hagan. Mucosal immunogenicity of plant lectins in mice. Immunology. 2000, 99(1), 30-37.
(12) W. Boonyo, H. E. Junginger, N. Waranuch, A. Polnok and T. Pitaksuteepong. Chitosan and trimethyl chitosan chloride (TMC) as adjuvants for inducing immune responses to ovalbumin in mice following nasal administration. J. Contr. Release., 2007, 121(3), 168-175.
(13) J. D. Bos, and M. K. Kapsenberg. The skin immune system: progress in cutaneous biology. Immunol. Today. 1993, 14, 75-78.
(14) E. van Wilsem, I. van Hoogstraten, J. Breve, Y. Zaman, and G. Kraal. In vivo antigen presentation capacity of dendritic cells from oral mucosa and skin draining lymph nodes. Adv. Exp. Med. Biol. 1994. 355, 63-67.
(15) G. M. Woods, K. G. Henderson, M. Qu and H. K. Mueller. Processing of complex antigens and simple hapten-like molecules by epidermal Langerhans cells. J. Leukoc. Biol. 1995. 57, 891-896.

1. An apparatus comprising:
a set of nano-sized projections carried by a carrier medium, the set of nano-sized projections shaped and configured to deliver a composition to a target site within a body;
a peripheral structure configured to receive the set of nano-sized projections at least partially therewithin; and
a displaceable carrier coupled to the peripheral structure and configured to be displaceable relative to the peripheral structure between a first position and a second position, the displacement of the displaceable carrier relative to the peripheral structure one of facilitating, effectuating and controlling a corresponding displacement of the set of nano-sized projections for inserting the set of nano-sized projections into the body.

2. The apparatus as in claim 1, wherein the set of nano-sized projections is couplable to the displaceable carrier, the displaceable carrier when disposed at the first position disposes the set of nano-sized projections at a retracted position that is within the peripheral structure, and the displaceable carrier when disposed at the second position disposes the set of nano-sized projections at an extended position that is at least partially external to the peripheral structure for enabling the insertion of the set of nano-projections into the body.

3.-4. (canceled)

5. The apparatus as in claim 1, further comprising a displacement control element coupled to the displaceable carrier, the displacement control element configured to control distance of displacement of the displaceable carrier relative to the peripheral structure, wherein a distance of displacement of the set of nano-sized projections relative to the peripheral structure is partially determined by a height of the peripheral structure.

6.-8. (canceled)

9. The apparatus as in claim 1, further comprising a set of force management elements coupled to the displaceable carrier, the set of force management elements configured to at least one of control, distribute, and limit a force corresponding to displacement of the set of nano-sized projections and facilitate insertion of the set of nano-sized projections into the body at a uniform at least one of pressure and distance.

10.-11. (canceled)

12. The apparatus as in claim 9, wherein the set of force management element is disposed within the peripheral structure.

13. The apparatus as in claim 12, wherein the set of force management elements comprises at least one spring coupled to the displaceable carrier, the at least one spring configured to at least one of control and distribute a force transferred from the displaceable carrier to the set of nano-sized projections to thereby one of facilitate and effectuate control of the insertion of the set of nano-sized projections into the body.

14. The apparatus as in claim 1, wherein a number of nano-sized projections within the set of nano-sized projections comprise segmented nano-sized projection having at least two segments that are stacked relative to each other.

15. (canceled)

16. The apparatus as in claim 14, wherein segmented nano-sized projections comprise a first end disposed distal to the carrier medium and a second end disposed proximal to the carrier medium, and wherein one of the diameter of the first end of the segmented nano-sized projections is at least approximately 10% smaller than the diameter of the second end of the segmented nano-sized projections, and wherein a distance between adjacent first ends of the nano-sized projections is between approximately 0.1 μm and 1.5 μm and a distance between adjacent second ends of the nano-sized projections is between approximately 0.05 μm and 0.75 μm.

17.-21. (canceled)

22. The apparatus as in claim 1, wherein the length of the nano-sized projections is between approximately 5 μm and 200 μm.

23.-24. (canceled)

25. The apparatus as in claim 22, wherein the density of the nano-sized projections carried by the carrier medium is at least approximately 100 nanoneedles/mm².

26.-27. (canceled)

28. The apparatus as in claim 1, wherein at least a portion of nano-sized projections of the set of nano-sized projections comprises at least one of solid nano-sized projections and hollow nano-sized projections that comprise a channel formed therewithin, the channel configured to allow communication of the composition therethrough, and wherein the apparatus further comprises a fluidic reservoir configured to hold a volume of the composition, the fluidic reservoir configured to be fluidly communicable with the channel of each hollow nano-sized projection such that the composition is communicable from the fluidic reservoir through the channel of each hollow nano-sized projection for delivery to the target site.

29.-32. (canceled)

33. The apparatus as in claim 1, wherein the set of nano-sized projections further comprises at least two nano-sized projection arrays, each nano-sized projection array comprising at least one nano-sized projections carried by a carrier medium, wherein the peripheral structure comprises
a set of peripheral structures configured to receive at least partially the at least two nano-sized projection arrays, and
wherein the displaceable carrier comprises a set of displaceable carriers coupled to the set of peripheral structures, the set of displaceable carriers couplable to the at least two nano-sized projection arrays and configured to be displaceable between a first position and a second position relative to the peripheral structure for displacing the at least two nano-sized projection arrays coupled thereto.

34.-40. (canceled)

41. The apparatuses as in claim 33, further comprising a connecting structure configured to interconnect the set of peripheral structures and the set of displaceable carriers, the connecting structure configured to at least one of control and distribute a force applied thereto to each displaceable carrier of the set of displaceable carriers and each corresponding nano-sized projection array carried by said displaceable carrier.

42.-46. (canceled)

47. A method for manufacturing an apparatus that is configured to deliver a composition to a target site within a body, the method comprising:
forming a set of nano-sized projections supported by a carrier medium, the set of nano-sized projections shaped and configured for delivering the composition to the target site;
disposing the set of nano-sized projections at least partially within a peripheral structure; and
coupling a displaceable carrier to the peripheral structure, the displaceable carrier couplable to the set of nano-sized projections and configured to be displaceable between a first position and a second position relative to the peripheral structure to thereby one of facilitate and effectuate a corresponding controlled displacement of the set of nano-sized projections for inserting the set of nano-sized projections into the body.

48. (canceled)

49. The method as in claim 47, wherein a disposition of the displaceable carrier at the first position relative to the peripheral structure disposes the set of nano-sized projections at a retracted position that is within the peripheral structure and a disposition of the displaceable carrier at the second position relative to the peripheral structure disposes the set of nano-sized projections at an extended position that is at least partially external to the peripheral structure.

50. (canceled)

51. The method as in claim 47, further comprising providing a displacement control element configured to control a distance of displacement of the displaceable carrier relative to the peripheral structure.

52. The method as in claim 47, further comprising coupling a set of force management elements to the displaceable carrier, the set of force management elements configured to at least one of control, distribute, and limit a force corresponding to displacement of the displaceable carrier relative to the peripheral structure.

53.-54. (canceled)

55. The method as in claim 47, wherein at least a portion of the nano-sized projections are segmented nano-sized projections, forming of the segmented nano-sized projections comprising at least two synthesis steps, each synthesis step producing one nano-sized projection segment of the segmented nano-sized projections, the at least two nano-sized projection segments formed in the at least two synthesis steps are stacked relative to each other to thereby form at least a portion of the segmented nano-sized projections.

56. The method as in claim 55, wherein the segmented nano-sized projections has a generally tapered shape, each segmented nano-sized projection comprising a first end disposed distal the carrier medium and a second end disposed proximal the carrier medium, wherein a diameter of the first end is at least approximately 10% smaller than a diameter of the second end.

57. (canceled)

58. The method as in claim 47, further comprising attaching a sealing film to the peripheral structure to isolate the set of nano-sized projections that is disposed within the peripheral structure.

59. The method as in claim 47, wherein at least a portion of the set of nano-sized projections comprises a channel formed therein, the channel configured to allow communication of the composition therethrough, the method further comprising disposing a base substrate with a fluidic reservoir formed therewithin adjacent the displaceable carrier, the fluidic reservoir configured to store a volume of the composition and fluidically coupled to the at least a portion of the set of nano-sized projections comprising a channel formed therein.

60. (canceled)

61. The method as in claim 47, further comprising coating a surface of the set of nano-sized projections with a volume of the composition, the composition coated on the surface of the set of nano-sized projections deliverable to the target site when the set of nano-sized projections is inserted into the body.

62.-63. (canceled)

64. The method as in claim 47, wherein the set of nano-sized projections comprises zinc oxide and the carrier medium is made at least substantially from one of silicon and PDMS.

65. A method comprising:
forming a plurality of segmented nano-sized projections supported by a carrier medium, wherein each of the plurality of segmented nano-sized projections comprises at least two segments that are stacked relative to each other, and wherein at least a portion of the plurality of segmented nano-sized projections has a generally layered shaped; and
configuring the plurality of segmented nano-sized projections to be displaceable for one of facilitating and effectuating delivery of a composition to a target site at a uniform at least one of distance and pressure.

66. (canceled)

67. The method as in claim 65, further comprising:
disposing the plurality of segmented nano-sized projections within a peripheral structure; and
coupling a displaceable carrier to the peripheral structure, the displaceable carrier couplable to the plurality of segmented nano-sized projections and configured to be displaced relative to the peripheral structure to thereby displace the plurality of nano-sized projections for insertion into the body.

68. (canceled)

69. The method as in claim 67, further comprising coupling a set of force management elements to the displaceable carrier, the set of force management element configured to at least one of control, distribute, and limit a force applied to at least one of the displaceable carrier and the plurality of nano-sized projections.

70.-72. (canceled)

73. The method as in claim 47, wherein forming a set of nano-sized projection arrays comprises
forming at least two nano-sized projection arrays, each nano-sized projection array comprising at least one of nano-sized projections that is carried by a carrier medium, and wherein a disposition of the displaceable carrier at the first position disposes the nano-projection array at a retracted position located within the peripheral structure and a disposition of the displaceable carrier at the second position disposes the nano-projection array at an extended position located at least partially external to the peripheral structure to thereby one of facilitate and effectuate insertion of the set of nano-sized projections of said nano-projection array into the body.

74.-78. (canceled)

79. A method for transporting a composition to a target site, the method comprising:
providing a nano-sized projection array, the nano-sized projection array comprising a set of nano-sized projections and a carrier medium carrying the set of nano-sized projections, the nano-sized projection array disposed at least partially within a peripheral structure and carried by a displaceable carrier; and
displacing the displaceable carrier relative to the peripheral structure, wherein displacement of the displaceable carrier structure relative to the peripheral structure one of facilitates and effectuates an application of force onto the nano-sized projection array carried by the displaceable structure to thereby displace the nano-sized projection array for transporting the composition to the target site.

80. The method as in claim 79, further comprising displacing the displaceable carrier from a first position to a second position relative to the peripheral structure to thereby displace the nano-sized projection array carried by the displaceable carrier from a retracted position that is within the peripheral structure to an extended position that is at least partially exterior to the peripheral structure, respectively, to thereby one of facilitate and effectuate transport of the composition to the target site.

81.-82. (canceled)

83. The method as in claim 79, further comprising at least one of controlling, distributing, and limiting a force applied to at least one of the displaceable carrier and the nano-sized projection array using at least one of a displacement control element and a set of force management elements configured for displacement of the nano-sized projections towards the target site at a uniform at least one of pressure and depth.

84. (canceled)

85. The method as in claim 83, comprising coating at least a portion of the nano-sized projection array with a volume of the composition, wherein the set of nano-sized projections of the nano-sized projection array comprises solid nano-rods.

86. The method as in claim 83, wherein at least a portion of the nano-projections of the set of nano-projections includes a channel formed therethrough, the channels of the portion of nano-projections configured to allow communication of the composition therethrough.

87. (canceled)

88. The method as in claim 87, further comprising communicating the composition from a fluidic reservoir through the channels of the portion of nano-projections one of during and subsequent the displacement of the set of nano-projections to the target site for effectuating the transport of the composition to the target site.

89. The method as in claim 81, wherein the target site is a site within the skin of an organism and the composition is a vaccine.

90. The method as in claim 81, wherein the control of the displacement of the displaceable carrier relative to the peripheral structure one of facilitates and effectuates insertion of the nano-sized projections into a body by a distance that corresponds to the epidermis.

91. The method as in claim 90, wherein the control of the displacement of the displaceable carrier relative to the peripheral structure one of facilitates and effectuates displacement of the nano-sized projections into the body by a distance that prevents contact of the nano-sized projections with the dermis.

92-103. (canceled)
